(19) United States
(12) Patent Application Publication
(10) Pub. No.: US 2025/0066504 A1
(43) Pub. Date: Feb. 27, 2025

(54) USE OF OREGOVOMAB AND PARP INHIBITORS FOR TREATMENT OF OVARIAN CANCER

(71) Applicant: CANARIABIO INC., Pyeongtaek si, Gyeonggi-do (KR)

(72) Inventors: Christopher F. NICODEMUS, Charlestown, MA (US); Ragupathy MADIYALAKAN, Edmonton (CA)

(21) Appl. No.: 18/847,463

(22) PCT Filed: Mar. 16, 2023
(86) PCT No.: PCT/CA2023/050344

§ 371 Date: Sep. 16, 2024

Related U.S. Application Data
(60) Provisional application No. 63/320,844, filed on Mar. 17, 2022.

(51) Int. Cl. C07K 16/30; A61K 31/454; A61K 39/00

(52) U.S. Cl. CPC C07K 16/3092; A61K 31/454; A61K 2039/545

(57) ABSTRACT

The present document describes a method for improving likelihood of survival in a stage III-IV ovarian cancer patient. The method comprises at least steps of (a) administering daily to a stage III-IV ovarian cancer patient a therapeutically effective dose of Niraparib, for at least 12 weeks; and (b) administering to the patient a therapeutically effective dose of monoclonal antibody mAb-B43.13 during week 1, week 4, week 7, week 12 and week 20 of a treatment period, wherein (a) is performed first and step (b) is performed second, to increase the patient's likelihood of survival in comparison with a control patient who has been diagnosed with stage III-IV ovarian cancer and has received standard of care chemotherapy treatment.

### CROSS-REFERENCE TO RELATED APPLICATIONS

This application claims priority under 35USC§ 119(e) of U.S. provisional patent application 63/320,844 filed on Mar. 17, 2022, the specification of which is hereby incorporated by reference in its entirety.

### BACKGROUND

### (a) Field

The subject matter disclosed generally relates to method for improving the likelihood of survival in a stage III-IV ovarian cancer patient by administering combination immunotherapy comprising a monoclonal antibody and a PARP inhibitor, more particularly the combination of monoclonal antibody mAb-B43.13 (oregovomab) and the PARP inhibitor Niraparib.

### (b) Related Prior Art

Ovarian cancer is a malignant tumor of the ovary and is the second most frequent cancer of the sexual genital organs after cervical cancer. The most common type of ovarian cancer is epithelial ovarian cancer, which occurs in the epithelial cells that cover the outer layer of the ovary, and accounts for about 90% of all ovarian cancer. Since there are no specific symptoms, more than 60% of cases are already at stage 3 or higher at the time of diagnosis, and most tend to occur simultaneously in both ovaries [Matsuo, K. et al. (2010); Hamanishi J., et al. (2015)]. It is a reality that the possibility of recurrence is high because current ovarian cancer surgery or standard cytotoxic chemotherapy cannot completely cure the cancer. Ovarian cancer surgery aims to remove as many cancer cells as possible in the body regardless of the disease progression stage, but ovarian cancer has small cancer cells and a fast metastasis, making it difficult to completely remove cancer cells during surgery. Also, anticancer drugs that are currently used cannot completely remove cancer cells. Some of the ovarian cancer cells have anticancer drug resistance. Thus, even after chemotherapy, some do not disappear and continue to grow, so many patients experience anticancer drug resistance or recurrence. In addition, target treatments developed to compensate the limitations of cytotoxic anticancer drugs have limitations in preventing frequent recurrences as they only delay recurrence by 4 months compared to standard anticancer treatments [Burger R A, et al. (2011); Perren T J, et al. (2015)].

Oregovomab is a murine IgG1 monoclonal antibody (mAb) with a K-light chain. It has a binding force of less than nano units to cancer antigen 125 (CA-125) during blood circulation. The therapeutic purpose of oregovomab administration is that it plays an important role in the recognition of the CA-125 antigen to lymphocytes by antigen-processing cells, especially dendritic cells (FIG. 1). This is a mechanism similar to that of other anticancer vaccines [Berek J, et al. (2009)]. CA-125 is a large membrane glycoprotein known as mucin 16 or MUC16, and is encoded by the MUC16 gene. CA-125 is one of the known tumor-associated antigens (TAA) for ovarian cancer and is well expressed against epithelial cancer cells. CA-125 is detected at a high frequency in the blood of more than 80% of patients with epithelial ovarian cancer, and this tumor-associated antigen (CA-125) increases with cancer progression. CA-125 is a proven tumor marker used to evaluate treatment response as well as predict disease recurrence. Fundamental studies that have employed oregovomab have generated several studies characterizing the induction of tumor-specific immunity. In a severe complex immunodeficiency mice model with CA-125-expressing tumors, the administration of oregovomab inhibited ascites production and reduced tumor size, leading to long-term survival. The most potent biological mechanism of oregovomab is to activate T helper (Th)1 cells or cytotoxic T lymphocyte (CTL) cells by allowing dendritic cells to express the antigen for CA-125, thereby promoting anti-cancer effects against CA-125 overexpressed carcinomas. More than 90% of patients generated an antibody response to the immunoglobulin constant region regarding oregovomab, and an antibody response was also induced in the oregovomab variable domain portion. The administration of oregovomab forms an immune antibody complex (CA-125-oregovomab complex) in the blood as well as in the peripheral tissues. The induction of this immune antibody complex induces a therapeutic immune response [Brewer, M., et al. (2020)].

Poly (ADP-ribose) polymerase (PARP), specifically PARPs 1 and 2, are zinc-finger deoxyribonucleic acid (DNA)-binding enzymes that play a crucial role in DNA repair (Fong et al. 2009). Upon formation of DNA breaks, PARP binds at the end of broken DNA strands, a process that activates its enzymatic activity.

Activated PARP catalyzes the addition of long polymers of adenosine diphosphate (ADP)-ribose onto PARP and several other proteins associated with chromatin, including histones and various DNA repair proteins (De Lorenzo et al. 2013, Jones et al. 2015). This results in chromatin relaxation, fast recruitment of DNA repair proteins, and efficient repair of DNA breaks. In this manner, PARP plays a key role in sensing DNA damage and converting it into intracellular signals that activate the base excision repair (BER) and single-strand break repair pathways.

Normal cells repair up to 10,000 DNA defects daily, and single-strand breaks are the most common form of DNA damage. Cells that are unable to repair this burden of DNA damage, such as those with defects in the homologous recombination or BER pathways, are at risk for accumulating multiple lesions that will ultimately trigger apoptosis. They enter the S phase (DNA replication) of the cell cycle with unrepaired single- and double-strand breaks. Pre-existing single-strand breaks are converted to double-strand breaks as the replication machinery passes. Accumulated double-strand breaks present during S phase are repaired by homologous recombination. Homologous recombination is the preferred repair pathway because it is associated with a much lower error rate than other forms of repair.

Cells that are unable to perform DNA repair via homologous recombination (e.g., due to inactivation of genes required for homologous recombination, such as breast cancer [BRCA1] or breast cancer 2 [BRCA2]-mutated cells), are at risk for accumulating multiple lesions that will ultimately trigger apoptosis. These cells accumulate stalled replication forks during S phase and are more likely to use the error-prone non-homologous end joining (NHEJ) or alternative (alt)-NHEJ pathways to repair double-strand breaks in DNA. Accumulation of errors in DNA by NHEJ contributes to mutation burden that promotes the development of cancer. Over time, the buildup of excessive DNA errors in combination with the inability to complete S phase because of stalled replication forks, contributes to cell death (De Lorenzo et al. 2013, Jones et al. 2015). In this way, treatment with PARP inhibitors represent a novel opportunity to selectively kill cancer cells. For example, a tumor arising in a patient with a BRCA mutation (BRCAm) has a defective homologous recombination DNA repair pathway and would be increasingly dependent on NHEJ, alt-NHEJ, and BER for maintenance of genomic integrity. PARP inhibitors block alt-NHEJ and BER, forcing tumors with BRCA deficiencies to use the error-prone NHEJ to fix double-strand breaks (Fong et al. 2013). Non-BRCA deficiencies in homologous recombination DNA repair genes could also enhance tumor cell sensitivity to PARP inhibitors (Gelmon et al. 2011). The rationale for anti-cancer activity in a subset of non-BRCAm tumors is that they share distinctive DNA repair defects with BRCAm carriers, a phenomenon broadly described as “BRCAness” (Turner et al. 2004). DNA repair defects can be caused by germline or somatic alterations to the homologous recombination DNA repair pathway. In a recent analysis of approximately 500 high-grade serous ovarian adenocarcinoma tumors, approximately 50% contained HRD (CGA 2011). A subset of these tumors had biologically plausible molecular alterations that may make them sensitive to PARP inhibition by niraparib. A similar analysis of triple-negative breast cancer indicates that 43% to 44% of these subjects have tumors with HRD (Timms et al. 2014). Homologous recombination is a complex pathway, and several genes other than BRCA1 and BRCA2 are required either to sense or repair DNA double-strand breaks via the homologous recombination pathway. Therefore, PARP inhibitors are also selectively cytotoxic for cancer cells with deficiencies in DNA repair proteins other than BRCA1 and BRCA2 (Turner et al. 2011). Recent clinical studies have shown PARP inhibitors to be active in breast and ovarian cancer, and potentially other tumor types (Pilié et al. 2019) Clinical anti-cancer activity with PARP inhibitors has been seen in both subjects with BRCAm and without BRCAm; however, activity is more robust in subjects with the germline mutation. (Kummar et al. 2012)

In summary, treatment with PARP1/2 inhibitors represents an opportunity to selectively kill a subset of cancer cell types by exploiting their deficiencies in DNA repair. Human cancers exhibit genomic instability and an increased mutation rate due to underlying defects in DNA repair. These deficiencies render cancer cells more dependent on the remaining DNA repair pathways, and targeting these pathways is expected to have a much greater impact on the survival of the tumor cells than that of normal cells.

Niraparib is a potent, orally active PARP1 and PARP2 inhibitor being developed as a treatment and maintenance therapy for subjects with tumors that harbor defects in the homologous recombination DNA repair pathway or that are driven by PARP-mediated transcription factors and is also being evaluated in subjects who have HRD negative and non-BRCA-mutated cancers.

Therefore, there is a need for administration of oregovomab, which can selectively recognize ovarian cancer, and PARP inhibitor therapy.

### SUMMARY

According to an embodiment there is provided a method for improving likelihood of survival in a stage III-IV ovarian cancer patient, the method comprising at least steps (a) and (b):
(a) administering daily to a stage III-IV ovarian cancer patient a therapeutically effective dose of Niraparib, for at least 12 weeks;
(b) administering to the patient a therapeutically effective dose of monoclonal antibody mAb-B43.13 during week 1, week 4, week 7, week 12 and week 20 of a treatment period,
wherein step (a) is performed first and step (b) is performed second,
thereby increasing the patient's likelihood of survival in comparison with a control patient who has been diagnosed with stage III-IV ovarian cancer and has received standard of care chemotherapy treatment.

The therapeutically effective dose of Niraparib may be from about 200 to about 300 mg, or is 200 mg or is 300 mg.

The therapeutically effective dose of Niraparib may be orally administered.

The therapeutically effective dose of monoclonal antibody mAb-B43.13 may be administered on day 1 of the week 1, week 4, week 7, week 12 and week 20 of the treatment period.

The treatment period may be 24 weeks.

2 mg of mAb-B43.13 may be administered.

The mAb-B43.13 may be administered in a volume of 50 ml by a 20-minute infusion.

According to another embodiment there is provided a therapeutic agent for use in inhibiting stage III-IV ovarian cancer tumor growth in a patient, the therapeutic agent comprising mAb-B43.13 (oregovomab) and a parp inhibitor;
wherein said parp inhibitor is Niraparib;
wherein said therapeutic agent is used according to the following schedule:
   (a) daily use by a stage III-IV ovarian cancer patient of a therapeutically effective dose of Niraparib, for at least 12 weeks;
   (b) use by the patient of a therapeutically effective dose of monoclonal antibody mAb-B43.13 during week 1, week 4, week 7, week 12 and week 20 of a treatment period,
wherein step (a) is first and step (b) is second,
thereby increasing the patient's likelihood of survival in comparison with a control patient who has been diagnosed with stage III-IV ovarian cancer and has received standard of care chemotherapy treatment.

The therapeutically effective dose of Niraparib may be from about 200 to about 300 mg, or is 200 mg or is 300 mg.

The therapeutically effective dose of Niraparib may be through oral use.

The therapeutically effective dose of monoclonal antibody mAb-B43.13 may be used on day 1 of the week 1, week 4, week 7, week 12 and week 20 of the treatment period.

The use period may be 24 weeks.

2 mg of mAb-B43.13 may be used.

The mAb-B43.13 may be used in a volume of 50 ml by a 20-minute infusion.

According to another embodiment there is provided a use of a therapeutic agent in inhibiting stage III-IV ovarian cancer tumor growth in a patient, the therapeutic agent comprising mAb-B43.13 (oregovomab) and a parp inhibitor;
wherein said parp inhibitor is Niraparib;
wherein said therapeutic agent is used according to the following schedule:
   (a) daily use by a stage III-IV ovarian cancer patient of a therapeutically effective dose of Niraparib, for at least 12 weeks;
   (b) use by the patient of a therapeutically effective dose of monoclonal antibody mAb-B43.13 during week 1, week 4, week 7, week 12 and week 20 of a treatment period,
wherein step (a) is first and step (b) is second,
thereby increasing the patient's likelihood of survival in comparison with a control patient who has been diagnosed with stage III-IV ovarian cancer and has received standard of care chemotherapy treatment.

The therapeutically effective dose of Niraparib may be from about 200 to about 300 mg, or is 200 mg or is 300 mg.

The therapeutically effective dose of Niraparib may be through oral use.

The therapeutically effective dose of monoclonal antibody mAb-B43.13 may be used on day 1 of the week 1, week 4, week 7, week 12 and week 20 of the treatment period.

The use period may be 24 weeks.

2 mg of mAb-B43.13 may be used.

The mAb-B43.13 may be used in a volume of 50 ml by a 20-minute infusion.

The following terms are defined below.

The terms “administration of” and/or “administering a” is intended to mean providing an antibody, chemotherapy, and/or their combination according to the present invention to a subject in need of treatment.

The term “composition” intended to mean a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the pharmaceutically acceptable carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing an antibody according to the present invention and a pharmaceutically acceptable carrier. By “pharmaceutically acceptable” it is meant the carrier, diluent or excipient must be compatible with the other ingredients of the formulation and not deleterious to the recipient thereof.

The term “chemotherapy regimen” is intended to mean combination of several chemotherapeutic agents. The rationale behind such chemotherapy regimen is that different chemotherapy drugs work through different cytotoxic mechanisms, and that the results of using multiple drugs will be synergistic to some extent. Because they have different dose-limiting adverse effects, they can be given together at full doses in chemotherapy regimens. Chemotherapy regimen may include induction and maintenance regimen.

The term “induction regimen” is intended to mean a chemotherapy regimen used for the initial treatment of a disease.

The term “maintenance regimen” is intended to mean the ongoing use of chemotherapy to reduce the chances of a cancer recurring or to prevent an existing cancer from continuing to grow.

In some embodiments, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the antibody or fragment thereof, preferably in purified form, together with a suitable amount of carrier so as to provide the form for proper administration to the patient. The formulation should suit the mode of administration.

The terms “inhibit”, “inhibition” or “inhibiting” as used herein in the context of the invention means to slow, hinder, restrain reduce or prevent. For example, “inhibiting growth” of a tumor cell as that term is used herein means to slow, hinder, restrain, reduce or prevent the tumor cell from growing.

The term “administering” as used herein refers to any action that results in exposing or contacting a composition containing the therapeutic monoclonal antibodies of the present invention in combination with the disclosed chemotherapy regimen. As used herein, administering may be conducted in vivo, in vitro, or ex vivo. Particularly, administering is to an ovarian cancer patient, for example a stage III-IV ovarian cancer patient, and more specifically, stage III-IV ovarian cancer patient that are BRCA-wild type that are platinum sensitive. For example, a composition may be administered by injection or through an endoscope. Administering also includes the direct application to cells of a composition according to the present invention. For example, during the course of surgery, tumor cells may be exposed. In accordance with an embodiment of the invention, these exposed cells (or tumors) may be exposed directly to a composition of the present invention, e.g., by washing or irrigating the surgical site and/or the cells, or by direct intra-tumoral injection of the therapeutic monoclonal antibody specific for a tumor associated antigen in combination with at least one immunostimulatory compound, and at least one immune homeostatic checkpoint inhibitor individually or in a mixture.

The term “subject” as used herein, is a human patient or other animal such as another mammal with functional mast cells, basophils, neutrophils, eosinophils, monocytes, macrophages, dendritic cells, and Langerhans cells. In humans, the appropriate cells express the high affinity receptor for IgG for the administered IgG antibody of the invention, as well as IgE (FcεRI) for the administered IgE antibody of the invention. Particularly, the subject may be an ovarian cancer patient, for example a stage III-IV ovarian cancer patient, and more specifically, stage III-IV ovarian cancer patient that are BRCA-wild type that are platinum sensitive.

As used herein, a reduction in growth kinetics, or complete elimination of, a cancer tumor or a metastasized cell or tumor as used herein is defined to mean that which is as understood in the art. For example, a reduction in growth kinetics means a reduction in the exponential growth, specific growth rate, or doubling time of a primary solid tumor, metastasized cell, or metastasized tumor relative to the exponential growth, specific growth rate, or doubling time normally observed in vivo or in vitro for a given tumor type. Complete elimination of a tumor is the absence of tumor presence, either by symptoms, physical exam, or radiographic imaging, in the presence of the therapeutic monoclonal antibody specific for a tumor associated antigen in combination with at least one immunostimulatory compound, and at least one immune homeostatic checkpoint inhibitor, where a tumor was previously seen to be present by these detection methodologies.

The term “time sufficient for treatment” or is intended to mean any period of time suitable to effect treatment with the immune adjuvant. In embodiments, that time period may be the time of a cycle used in standard to care for the immune adjuvant (e.g., chemotherapy). Examples of standard of care treatments may be found for example in Gynecologic Oncology Group Chemotherapy Procedures Manual, incorporated herein by reference. The length of chemotherapy treatment is determined by a variety of factors. These include the type of cancer, the extent of cancer, the types of drugs that are given, as well as the expected toxicities of the drugs and the amount of time necessary to recover from these toxicities. Many chemotherapy treatment schedules (often referred to as Standard of Care (SOC), including the type and length of chemotherapy treatment) have been determined through clinical trials that compared them and determined which had the most benefit and was most well tolerated. In general, chemotherapy treatment is given in cycles. This allows the cancer cells to be attacked at their most vulnerable times, and allows the body's normal cells time to recover from the damage. There are really three issues regarding the cycle time, duration of the cycle, frequency of the cycle, and how many cycles. Duration of the cycle: chemotherapy treatment may be a single drug or a combination of drugs. The drugs may all be given on a single day, several consecutive days, or continuously as an outpatient or as an inpatient. Treatment could last minutes, hours, or days, depending on the specific protocol. Frequency of the cycle: chemotherapy may repeat weekly, bi-weekly, or monthly. Usually, a cycle is defined in monthly intervals. For example, two bi-weekly chemotherapy sessions may be classified as one cycle. The number of cycles: In most cases, the number of cycles—or the length of chemotherapy from start to finish—has been determined by research and clinical trials. When cure is the treatment goal. Adjuvant chemotherapy (therapy after surgery has removed all visible cancer) may last 4-6 months. Adjuvant chemotherapy is common in cancers of the breast and colon. In cancers of the testis, Hodgkin and non-Hodgkin lymphoma, and leukemias, length of chemotherapy treatment may be up to a year. When there is visible disease, the length of chemotherapy treatment will depend upon the response of the disease to therapy. If the disease disappears completely, chemotherapy may continue for 1-2 cycles beyond this observation to maximize the chance of having attacked all microscopic disease. If the disease shrinks but does not disappear, chemotherapy will continue as long as it is tolerated and the disease does not grow. If the disease grows, the chemotherapy will be stopped. As patients experience toxicities and blood cell counts, the actual timing of the cycles is sometimes delayed according the necessities of each patient's circumstance. Depending on the health and wishes of the patient, either different drugs may be given to try to kill the cancer, or chemotherapy will be stopped and the goal changed to focus on patient comfort.

The term “PARP inhibitor(s)” is intended to mean the group of pharmacological inhibitors of the enzyme poly ADP ribose polymerase (PARP). PARP inhibitors that are approved for marketing include Olaparib, Rucaparib, Niraparib, and Talazoparib. PARP inhibitors under trial include for example Talazoparib, Veliparib, Pamiparib, Rucaparib, Veliparib.

The term “standard of care chemotherapy treatment” is intended to mean the type and length of chemotherapy treatment received by stage III-IV ovarian cancer patients as the current treatment accepted as a standard, which involves for example several cycles of paclitaxel and carboplatin treatment, and does not involve immunotherapy.

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### DETAILED DESCRIPTION

The present invention is directed to a method for improving likelihood of survival in a stage III-IV ovarian cancer patient whereby the PARP inhibitor niraparib is used as a first line treatment, in combination with immunotherapy. The applicant believes that this is the first instance where niraparib is used as a first line therapy, prior to second line therapy, as all approvals of the compound, to date, are as a second line treatment post chemotherapy.

In embodiments there is disclosed a method for improving likelihood of survival in a stage III-IV ovarian cancer patient, the method comprising at least steps (a) and (b):
(a) administering daily to a stage III-IV ovarian cancer patient a therapeutically effective dose of Niraparib, for at least 12 weeks;
(b) administering to the patient a therapeutically effective dose of monoclonal antibody mAb-B43.13 during week 1, week 4, week 7, week 12 and week 20 of a treatment period,
wherein step (a) is performed first and step (b) is performed second,
thereby increasing the patient's likelihood of survival in comparison with a control patient who has been diagnosed with stage III-IV ovarian cancer and has received standard of care chemotherapy treatment.

The treatment period may be 24 weeks.

### Oregovomab

Oregovomab, also known as mAb-B43.13 is a murine IgG1 antibody specific to CA125 (MUC16). Antibody B43.13 was deposited under the Budapest Treaty in the ATCC, 1801 University Blvd., Manassas, Va. 20110-2209 and given the Patent Deposit Designation PTA-1883 on May 18, 2000. [R. Madiyalakan et al, Hybridoma, 14:199-203 (1995), incorporated herein by reference].

The therapeutically effective dose of monoclonal antibody mAb-B43.13 may be administered on day 1 of the week 1, week 4, week 7, week 12 and week 20 of the treatment period.

In embodiments, 2 mg of mAb-B43.13 may be administered. According to another embodiment, 1 mg of mAb-B43.13 may be administered. According to another embodiment, mAb-B43.13 may be administered in a volume of 50 ml by a 20-minute infusion.

### PARP Inhibitors

Poly (ADP-ribose) polymerase (PARP), specifically PARPs 1 and 2, are zinc-finger deoxyribonucleic acid (DNA)-binding enzymes that play a crucial role in DNA repair (Fong et al. 2009). Upon formation of DNA breaks, PARP binds at the end of broken DNA strands, a process that activates its enzymatic activity.

Treatment with PARP1/2 inhibitors represents an opportunity to selectively kill a subset of cancer cell types by exploiting their deficiencies in DNA repair. Human cancers exhibit genomic instability and an increased mutation rate due to underlying defects in DNA repair. These deficiencies render cancer cells more dependent on the remaining DNA repair pathways, and targeting these pathways is expected to have a much greater impact on the survival of the tumor cells than that of normal cells.

The term “PARP inhibitor(s)” is intended to mean the group of pharmacological inhibitors of the enzyme poly ADP ribose polymerase (PARP). PARP inhibitors that are approved for marketing include Olaparib, Rucaparib, Niraparib, and Talazoparib. PARP inhibitors under trial include for example Talazoparib, Veliparib, Pamiparib, Rucaparib, Veliparib.

According to an embodiment, the PARP inhibitor is Niraparib. It is a potent, orally active PARP1 and PARP2 inhibitor being developed as a treatment and maintenance therapy for subjects with tumors that harbor defects in the homologous recombination DNA repair pathway or that are driven by PARP-mediated transcription factors and is also being evaluated in subjects who have HRD negative and non-BRCA-mutated cancers.

In embodiments, the therapeutically effective dose of Niraparib may be from about 200 to about 300 mg, or 200 mg or 300 mg. In embodiments, the therapeutically effective dose of Niraparib may be orally administered.

According to another embodiment there is disclosed a therapeutic agent for use in inhibiting stage III-IV ovarian cancer tumor growth in a patient, the therapeutic agent comprising mAb-B43.13 (oregovomab) and a parp inhibitor. The parp inhibitor may be Niraparib. The therapeutic agent may be used according to the following schedule:
(a) daily use by a stage III-IV ovarian cancer patient of a therapeutically effective dose of Niraparib, for at least 12 weeks;
(b) use by the patient of a therapeutically effective dose of monoclonal antibody mAb-B43.13 during week 1, week 4, week 7, week 12 and week 20 of a treatment period,
wherein step (a) is first and step (b) is second,
thereby increasing the patient's likelihood of survival in comparison with a control patient who has been diagnosed with stage III-IV ovarian cancer and has received standard of care chemotherapy treatment.

According to an embodiment, the PARP inhibitor is Niraparib. It is a potent, orally active PARP1 and PARP2 inhibitor being developed as a treatment and maintenance therapy for subjects with tumors that harbor defects in the homologous recombination DNA repair pathway or that are driven by PARP-mediated transcription factors and is also being evaluated in subjects who have HRD negative and non-BRCA-mutated cancers.

In embodiments, the therapeutically effective dose of Niraparib may be from about 200 to about 300 mg, or 200 mg or 300 mg. In embodiments, the therapeutically effective dose of Niraparib may be orally administered.

The therapeutically effective dose of monoclonal antibody mAb-B43.13 may be administered on day 1 of the week 1, week 4, week 7, week 12 and week 20 of the treatment period.

In embodiments, 2 mg of mAb-B43.13 may be administered. According to another embodiment, 1 mg of mAb-B43.13 may be administered. According to another embodiment, mAb-B43.13 may be administered in a volume of 50 ml by a 20-minute infusion.

According to another embodiment there is disclosed a use of a therapeutic agent in inhibiting stage III-IV ovarian cancer tumor growth in a patient, the therapeutic agent comprising mAb-B43.13 (oregovomab) and a parp inhibitor;
wherein said parp inhibitor is Niraparib;
wherein said therapeutic agent is used according to the following schedule:
   (a) daily use by a stage III-IV ovarian cancer patient of a therapeutically effective dose of Niraparib, for at least 12 weeks;
   (b) use by the patient of a therapeutically effective dose of monoclonal antibody mAb-B43.13 during week 1, week 4, week 7, week 12 and week 20 of a treatment period,
wherein step (a) is first and step (b) is second,
thereby increasing the patient's likelihood of survival in comparison with a control patient who has been diagnosed with stage III-IV ovarian cancer and has received standard of care chemotherapy treatment.

According to an embodiment, the PARP inhibitor is Niraparib. It is a potent, orally active PARP1 and PARP2 inhibitor being developed as a treatment and maintenance therapy for subjects with tumors that harbor defects in the homologous recombination DNA repair pathway or that are driven by PARP-mediated transcription factors and is also being evaluated in subjects who have HRD negative and non-BRCA-mutated cancers.

In embodiments, the therapeutically effective dose of Niraparib may be from about 200 to about 300 mg, or 200 mg or 300 mg. In embodiments, the therapeutically effective dose of Niraparib may be orally administered.

The therapeutically effective dose of monoclonal antibody mAb-B43.13 may be administered on day 1 of the week 1, week 4, week 7, week 12 and week 20 of the treatment period.

In embodiments, 2 mg of mAb-B43.13 may be administered. According to another embodiment, 1 mg of mAb-B43.13 may be administered. According to another embodiment, mAb-B43.13 may be administered in a volume of 50 ml by a 20-minute infusion.

### Abbreviations

° C. degrees Celsius
° F. degrees Fahrenheit
ADCC Antibody-Dependent Cell-Mediated Cytotoxicity
AE Adverse Event
ALP Alkaline phosphatase
ALT Alanine Aminotransferase
AML Acute Myeloid Leukemia
ANC Absolute Neutrophil Count
AST Aspartate Aminotransferase
BOR Best Overall Response
BUN Blood Urea Nitrogen
CA125 Cancer Antigen 125
CBC Complete Blood Count
CFR Code of Federal Regulations
CI Confidence Interval
cm Centimeter
CRF Case Report Form
CSR Clinical Study Report
CT Computed Tomography
CTCAE Common Terminology Criteria for Adverse Events
DNA Deoxyribonucleic Acid
DCR Disease Control Rate
dBP Diastolic Blood Pressure
DOR Duration of Response
EC Ethics Committee
ECG Electrocardiogram
ECOG Eastern Cooperative Oncology Group
EDC Electronic Data Capture
ELISPOT Enzyme-linked immunosorbent spot assay
FDA Food and Drug Administration
FIGO International Federation of Gynecology and Obstetrics
gBRCAmut Germline BRCA mutation
g/dL Grams Per Deciliter
GCIG Gynecologic Cancer Intergroup
GCP Good Clinical Practice
GI Gastrointestinal
GOG Gynecological Oncology Group
HAMA Human Anti-Mouse Antibody
Hb Hemoglobin
HBV Hepatitis B
HIPAA Health Insurance Portability and Accountability Act of 1996
HIV Human Immunodeficiency Virus
HSRs Hypersensitivity Reactions
HR Hazard Ratio
HRD Homologous Recombination Deficiency
HRDpos Homologous Recombination Deficiency positive
HRDneg homologous recombination Deficiency negative
ICF Informed Consent Form
ICH International Conference on Harmonization
IEC Independent Ethics Committee
IgG1 Immunoglobulin G subclass 1
IRB Institutional Review Board
ITT Intent-to-Treat
IV Intravenous
kDa Kilodaltons
kg Kilogram
LDH Lactate Dehydrogenase
LTFU Long Term Follow Up
MAb Monoclonal Antibody
MAb-B43.13 IgG1 anti-CA125 Monoclonal Antibody (oregovomab)
MDS Myelodysplastic syndrome
MedDRA Medical Dictionary for Regulatory Activities
mg Milligram
mL Milliliter
mL/min Milliliters per Minute
MRI Magnetic Resonance Imaging
MS Multiple Sclerosis
MUC16 Mucin 16 (another name for CA125)
N Normal
NMLR Neutrophil Monocyte Lymphocyte Ratio
oregovomab Generic name for MAb-B43.13
OS Overall Survival
PFS Progression Free Survival
PD-1 Programmed Death-1
QD Once daily
RBC Red Blood Cell
SAS Software suite developed by the SAS Institute for advanced analytics
sBP Systolic Blood Pressure
SDRC Safety Data Review Committee
SGOT Serum Glutamic Oxaloacetic Transaminase
SLE Systemic Lupus Erythematosus
SOC Standard of Care
μL Microliter
ULN Upper Limit of Normal

The present invention will be more readily understood by referring to the following examples which are given to illustrate the invention rather than to limit its scope.

### Example 1

Phase 1B/2, Single Arm Clinical Trial to Evaluate the Safety and Activity of Oregovomab and Niraparib as a Combinatorial Immune Priming Strategy in Subjects with Platinum Sensitive Recurrent Ovarian Cancer

### Study Population:

Adult subjects with CA125-associated, advanced recurrent epithelial ovarian, fallopian tube or peritoneal carcinoma (FIGO Stage Ill/IV), previously treated with up to 3 prior lines of therapy, and platinum sensitive for ≥6 months after the most recent platinum-containing regimen prior to the start of study treatment. That is, the subject responded to prior platinum therapy with >6-month progression free interval until current progression.

### Number of Subjects to be Enrolled:

Approximately 15 subjects will be screened to enroll approximately 10 evaluable subjects. The Sponsor may, after a review of the available efficacy and safety data, amend the protocol to enroll up to 30 additional subjects for further evaluation of efficacy in this study. The decision to amend the protocol to enroll additional subjects will be based on observation of early humoral response in 5 out of 10 evaluable patients at Week 7, and/or a Disease Control Rate (DCR), defined as CR, PR or SD in 4 out of 10 evaluable patients at Week 12 disease assessment.

### Study Rationale:

This is a single arm Phase 1b/2 evaluation of the combination of oregovomab and niraparib in subjects who have been previously treated with 1 to 3 platinum-based chemotherapy and have platinum sensitive (relapse free duration of >6 months) ovarian, fallopian tube and primary peritoneal cancers.

The combination will be evaluated as a combinatorial immune priming strategy. Both agents, oregovomab and niraparib, have previously been used in combination with other anti-cancer treatments and demonstrated bioactivity in advanced ovarian cancer. However, they have not previously been combined with each other. Although somewhat marrow suppressive, PARP inhibition has been demonstrated to be compatible with immunotherapy (Konstantinopoulos et al. 2019). Resultant double stranded DNA (dsDNA fragments) from treatment with niraparib should induce some level of immunogenic tumor cell death. The dsDNA fragments have been shown to activate the STING (STimulator of INterferon Genes) sensing pathway of innate immunity resulting in an enhanced IFN-γ inflammatory signature. Activating STING could also potentially reignite immunologically inert tumors as has been shown in mouse models in other tumor types (Jing et al. 2017). In the case of the combination of oregovomab with niraparib in ovarian cancer, hypothetically, T cells which have been immune sensitized by oregovomab may be further activated and trafficked into tumor in the presence of niraparib mediated induction of STING. One of the objectives of the study is to prolong the disease-free interval prior to the next platinum based chemotherapy because prolonging the platinum free interval by immune priming with oregovomab and niraparib for 12 to 24 weeks may also improve responsiveness to subsequent platinum-based therapy.

This proposed study will assess the immunological and clinical activity, early humoral response, and DCR, of concomitant oregovomab and oral daily niraparib. This will be a preliminary investigation of the interaction of indirect immunization with oregovomab and concomitant niraparib therapy and the immune modulatory effects associated with PARP inhibition. The population of subjects with recurrent disease that have received at least 1 to 3 prior lines of second line chemotherapy and remain platinum sensitive, defined as ovarian cancer that comes back 6 or more months after platinum-based treatment, provides a reasonably convenient window to determine potential safety and anti-tumor activity of combination oregovomab and niraparib. Study subjects must be deemed to be clinically appropriate, in the judgment of the clinical investigator, to receive a 12-week delay before initiation of the next-line cancer therapy while they receive the experimental immune modifying treatment with oregovomab and niraparib under this protocol.

Since clinical response to the oregovomab and niraparib combination has not yet been investigated, all subjects will be treated for 12 weeks with oregovomab and niraparib therapy, then assessed for immunological and clinical activity and tumor response at 12 weeks, 12 more weeks of oral niraparib should be given. For subjects who have progressive disease at week 12 or earlier, or unable to tolerate niraparib due to high grade toxicity in the judgment of the investigator, their treatment should be switched to the alternative next line of cancer treatment. All subjects, regardless of choice of therapy after the first 12 weeks of oregovomab and niraparib, will be treated with one additional monotherapy dose of oregovomab at week 20. Clinical response will be assessed again at week 24.

The possibility that responses to subsequent cancer therapy may be enhanced by the long-standing consequences of the interaction between oregovomab immune stimulation, and PARP mediated anti-tumor effects and its recently recognized ability to boost the immune system will also be considered. Progression free survival (PFS) status obtained from long term follow up may provide additional preliminary information supportive of immune priming in this concomitant treatment strategy.

The doses to be tested in this study for each component have been selected previously through human clinical study. Establishing safety and observing for activity of the concomitant exposure will provide justification for further assessment in the frontline, as well as other recurrent disease settings.

In summary, this study will assess immunological, clinical activity and tolerability of the concomitant treatment in the setting of platinum sensitive recurrent ovarian cancer.

### Rationale for 2 mg Dose of Oregovomab:

The dose of oregovomab is optimal for use in immunization protocols. As oregovomab is dosed at immune stimulating and not pharmacologic doses, it is expected that the combination will be well tolerated, but this will be explored in this controlled clinical trial setting. The dose of oregovomab is in the optimal range to induce cellular immunity, which diminishes at higher doses of the antibody. Oregovomab immunization is provided 5 times over 20 weeks, with a 2 mg infusion delivered IV over 20 minutes±5 minutes at weeks 1, 4, 7, 12 and 20.

### Rationale for 300 mg Dose of Niraparib:

The 300 mg dose of niraparib is the optimal pharmacologic dose approved clinically for use in ovarian cancer, balancing enzyme inhibition relative to toxicity. This dose of niraparib is associated with toxicities not seen with oregovomab, most notably a bone marrow suppression (neutropenia, thrombocytopenia, and anemia) in 20-40% of subjects, and hypertension/hypertensive crisis seen in 9% of subjects under treatment for a period of up to two years. Although somewhat marrow suppressive, PARP inhibition has been demonstrated to be compatible with immunotherapy as reported in an initial report at ASCO 2017 (Friedlander, 2017) and which has also been seen in unpublished studies.

Recently the management of marrow toxicity has been simplified by reducing the dose from 300 mg to 200 mg orally per day for based on either baseline body weight or platelet count, which reduces adverse effect on platelet counts, but maintains clinical activity.

This dose reduction was based on the ENGOT-OV16/NOVA and PN001 studies, and a model classification tree approach, which showed that subjects with a baseline body weight <77 kg or baseline platelet count <150,000 μL had an approximately 30% grade 3/4 thrombocytopenia rate in the first 30 days of treatment. Further, the actual delivered dose in these subjects approximated a starting dose of 200 mg despite the intended delivery of a starting dose of 300 mg.

In this study, subjects will be treated with starting dose of niraparib at 300 mg (3 tablets of 100 mg each) niraparib. Subjects whose baseline weight is <77 kg or baseline platelet count is <150,000 μL will be treated with starting dose of niraparib 200 mg (2 tablets of 100 mg each of niraparib).

### Study Design:

A Phase 1b/2 single arm open label study to evaluate the combination of oregovomab and niraparib as a combinatorial immune priming strategy in subjects with platinum sensitive recurrent ovarian cancer.

The study will include a:

### Screening Period (Up to 28 Days):

After signing informed consent, subjects will be screened for eligibility during the period of 28 days immediately prior to starting study drug on Week 1 Day 1 visit. Once all eligibility criteria are met, the subjects will be enrolled and initiate therapy.

Treatment Period (about 24 Weeks):

All subjects will receive the combination of niraparib and oregovomab as follows:
Niraparib will be administered orally once daily starting at the first day of treatment (Day 1 Week 1). The daily dose of niraparib will be 300 mg (3×100 mg tablets) taken orally from Day 1 Week 1 to at least the end of Week 12. For subjects whose baseline weight is <77 kg or baseline platelet count is <150,000 μL the daily dose of niraparib will be 200 mg (2×100 mg tablets).
Oregovomab (2 mg) will be administered via IV infusion in 50 mL of normal saline over 20 minutes±5 minutes during clinic visits at Day 1 of Week 1, Week 4, Week 7, Week 12, and Week 20.
   Subjects will be observed for 1-2 hours after the end of the oregovomab infusion.

All clinical, safety and immunological evaluations will be conducted according to a defined schedule during the treatment period and the survival follow-up period (see Appendix A).

The treatment period is defined as time from the beginning of first study treatment to treatment period exit. Treatment period exit is defined as Week 24 or early termination.
Subjects who have progressive disease or unknown [PD/UNK] per RECIST v1.1, or unable to tolerate niraparib due to high grade toxicity in the judgment of the investigator at Week 12, should discontinue niraparib and be started on alternative next line chemotherapy of Physician's choice.
Subjects who have stable disease [SD], partial response [PR], or complete response [CR], as per RECIST v1.1, at Week 12, should continue to take niraparib.
All subjects, regardless of whether they stay on niraparib or not, are required to take the 5th dose of oregovomab at Week 20 visit.

### Post-Treatment Follow-Up Period:

Initial safety follow-up for 30 days after the last dose of the last treatment taken.

### Long Term Follow Up:

PFS and safety data, including information regarding any interval treatments, will be collected every three months [±1 week] for one year, until death, withdrawal of consent, loss to follow-up, or sponsor decision to close study, whichever comes first for up to 1 year from the end of the Treatment Period. These follow-up visits may be performed by telephone if a subject is unable to visit the site.

### Diagnosis and Main Criteria for Inclusion/Exclusion:

### Main Inclusion Criteria:

Subjects are eligible to be included in the study only if all of the following criteria apply:
1. Adult females (18 years old and older)
2. Subjects with CA125-associated recurrent epithelial adenocarcinoma of ovarian, fallopian tube or peritoneal origin.
3. Have one of the eligible histologic epithelial cell types: serous adenocarcinoma, endometrioid adenocarcinoma, undifferentiated carcinoma, clear cell adenocarcinoma, mixed epithelial carcinoma, transitional cell carcinoma, malignant Brenner's Tumor, or adenocarcinoma not otherwise specified (N.O.S.).
4. Previously treated FIGO Stage III and IV disease with up to 3 prior lines of therapy, including at least one platinum-based therapy. Each line of therapy should have been changed due to recurrence, progression, or toxicity. Maintenance therapy with bevacizumab, hormonal therapies and/or a PARP inhibitor is not considered a line of therapy.
5. Must have received 1 to 3 prior lines of chemotherapy including prior platinum-based chemotherapy for first line ovarian cancer, however they must be platinum sensitive, defined as ovarian cancer that comes back 6 or more months after a platinum-based treatment prior to the start of study treatment.
6. Must have medical assessment consistent with prognosis for an expected survival of at least 6 months and be clinically appropriate to receive a 12-week hiatus from any cytotoxic treatment according to the best clinical judgement of the treating investigator.
7. Must have had an elevated serum CA125 >50 units/mL measured at screening within 28 days of start of study treatment.
8. Must have measurable disease, including identification of marker lesions, by radiographic or physical criteria suitable for evaluation according to RECIST v1.1 for documentation of disease response or progression.
9. Must have a ECOG Performance Status of 0, 1 or 2.
10. Must have adequate organ function defined as:
   a. Absolute neutrophil count ≥1,500/μL
   b. Platelets ≥100,000/μL
   c. Hemoglobin 8 g/dL
   d. Total bilirubin ≤1.5×ULN (52.0 in subjects with known Gilberts syndrome) OR direct bilirubin s 1×ULN
   e. LDH, SGOT and SGPT<2.5 times upper limits of normal
   f. Albumin >3.5 g/dL
   g. Serum creatinine ≤1.5× upper limit of normal (ULN) or calculated creatinine clearance 30 mL/min using the Cockcroft-Gault equation
11. For women of childbearing potential, negative pregnancy test and willingness to avoid pregnancy by using a highly effective method of contraception from the first dose of study treatment to 60 days after last dose of study treatment.
12. Able to take oral medications.
13. Sign informed consent and authorization permitting release of personal health information.

### Main Exclusion Criteria:

Subjects are excluded from the study if any of the following criteria apply:
1. Subject must not be simultaneously treated in any other interventional clinical trial.
2. Subject must not have had major surgery ≤3 weeks prior to initiating protocol therapy and subject must have recovered from any surgical effects.
3. Subject must not have received investigational therapy ≤4 weeks, or within a time interval less than at least 5 half-lives of the investigational agent, whichever is shorter, prior to initiating protocol therapy.
4. Subject has had radiation therapy encompassing >20% of the bone marrow within 2 weeks; or any radiation therapy within 1 week prior to Day 1 of protocol therapy.
5. Subject must not have received a transfusion (platelets or red blood cells) s 2 weeks prior to first dose of study treatment.
6. Subject must not have received colony-stimulating factors (e.g., granulocyte colony-stimulating factor, granulocyte macrophage colony-stimulating factor, or recombinant erythropoietin) within 4 weeks prior initiating protocol therapy.
7. Subject has had any known Grade 3 or 4 anemia, neutropenia or thrombocytopenia due to prior chemotherapy that persisted >4 weeks and was related to the most recent treatment.
8. Subject must not have a serious, uncontrolled medical disorder, nonmalignant systemic disease, or active, uncontrolled infection or active infection causing fever. Examples include, but are not limited to, uncontrolled major seizure disorder, unstable spinal cord compression, superior vena cava syndrome, or any psychiatric disorder that prohibits obtaining informed consent. Subjects with chronic diseases that are well controlled (e.g., diabetes mellitus, hypertension [<150 sBP and <90 dBP]) are eligible.
9. Evidence of clinically significant cardiovascular and respiratory conditions including myocardial infarction within 1 year, uncontrolled or unstable angina, congestive heart failure (New York Heart Association Class Ill or IV), arrhythmia (Grade 2 or higher), chronic obstructive pulmonary disease, persistent asthma, or a history of asthma within 5 years.
10. Subject must not have any known history of myelodysplastic syndrome (MDS) or acute myeloid leukemia (AML).
11. Diagnosed or treated for another malignancy within 5 years before the first dose, or previously diagnosed with another malignancy and have any evidence of residual disease. Subjects with non-melanoma skin cancer or cervix carcinoma in situ are not excluded if they have undergone complete resection.
12. Subject must not have known, symptomatic brain or leptomeningeal metastases.
13. Have an active autoimmune disease (e.g., rheumatoid arthritis, SLE, ulcerative colitis, Crohn's Disease, MS, ankylosing spondylitis, thyroiditis) requiring continuing immune suppressive therapy.
14. Recognized immunodeficiency condition including cellular immunodeficiencies, hypogammaglobulinemia or dysgammaglobulinemia; subjects who have hereditary, or congenital immunodeficiency's (HIV infection, see below).
15. Chronically treated with systemic doses of immunosuppressive drugs such as cyclosporine, methotrexate, adrenocorticotropic hormone (ACTH) or immune suppressive monoclonal antibodies.
16. Chronic therapeutic corticosteroid use, defined as >5 days of prednisone or equivalent, with the exception of inhalers or those on a pre-planned steroid taper. (Note: Premedication with corticosteroids per institutional standard of care is allowed).
17. Any previous treatments with oregovomab.
18. Known allergy to murine proteins or hypersensitivity to any of the excipients of oregovomab or niraparib.
19. Have contraindications to the use of presser agents (e.g., SC epinephrine), notably monoamine oxidase inhibitor (MAOI) use.
20. Any of the following conditions (on-study testing is not required):
   a. Known HIV-infected subjects unless on effective anti-retroviral therapy with an undetectable viral load within 6 months, or
   b. Known or suspected hepatitis B if active infection (subjects with chronic hepatitis B infection must have an undetectable HBV viral load on suppressive therapy, if indicated; positive surface antibody alone is not an exclusion), or
   c. Known or suspected hepatitis C infection which has not been treated and cured unless currently on treatment with an undetectable viral load).

Unable to understand, and/or unwilling to sign a written consent form which must be obtained prior to treatment.

### Study Objectives and Endpoints:

| | |
|---|---|
| Objectives | Endpoints |

Primary

| | |
|---|---|
| Disease Control Rate (DCR) | Per RECIST v1.1 as determined by the |
| To evaluate anti-tumor activity | investigator: |
| of oregovomab added to | DCR, defined as the proportion of |
| niraparib at 12 and 24 weeks | subjects with complete response (CR), |
| in subjects with CA125- | partial response (PR) and stable disease |
| associated recurrent epithelial | (SD), at 12 and 24 weeks. |
| adenocarcinoma of ovarian, | Duration of Response, defined as the |
| fallopian tube or peritoneal | time measurement criteria are met for |
| origin. | SD, CR or PR until the first date that |
| | recurrence or PD is objectively |
| | documented. |

Secondary

| | |
|---|---|
| Safety and Tolerability | Frequency/severity of vital signs |
| To establish the preliminary | measurements, physical examination |
| safety and tolerability of | findings, changes in clinical laboratory |
| oregovomab added to niraparib | parameters (hematology, biochemistry, |
| in subjects with CA125- | urinalysis), and incidence of adverse |
| associated recurrent epithelial | events (AEs), serious AEs (SAEs), |
| adenocarcinoma of ovarian, | deaths, and AEs leading to |
| fallopian tube or peritoneal | discontinuation of treatment. |
| origin. | |
| Humoral immune responses | Antibody to oregovomab as measured |
| | by HAMA. |
| | Early humoral response measured at |
| | Week 7 (Day 43) relative to baseline is |
| | special interest and is evaluated serially |
| | through the treatment phase of the |
| | study. |

Exploratory

| | |
|---|---|
| To explore the effect of | Progression-Free Survival (PFS), |
| oregovomab added to | defined as date of first dose of study |
| niraparib with respect to initial | treatment to the date of event defined as |
| progression, response rate, | the first documented progression as per |
| time to subsequent therapies, | RECIST v1.1 as determined by the |
| time to next progression and | investigator or death due to any cause. |
| death. | Overall Survival (OS), defined as date |
| | of first dose of study treatment to date |
| | of death due to any cause. |
| | Overall Response Rate (ORR) to |
| | alternate next-line therapy-initiated post |
| | Week 12 measured at Week 24 relative |
| | to their Week 12 assessment (new |
| | baseline). |
| | Time to First Subsequent Therapy |
| | (TFST), defined as time from the date |
| | of first dose of study treatment to |
| | second anti-cancer therapy start date |
| | following study treatment completion, |
| | treatment exit [EOT] or death. |

Potential Immunological Responses/Biomarkers

| | |
|---|---|
| Genetic Status | Homologous Recombination |
| | Deficiency (HRD) status (patient |
| | history) |
| | BRCA 1 and BRCA2 (germline |
| | and somatic) (patient history) |

### Study Duration:

Subjects will be treated for approximately 24 weeks during the Treatment Period, enter Post-Treatment Follow Up Phase with an initial 30-day safety follow-up and then followed for survival until death, withdrawal of consent, lost to follow-up, sponsor decision to close study for up to 1 year after treatment exit or early termination. It is anticipated that recruitment and treatment will be completed within approximately 12 months and the entire study, including follow up period, will last approximately 2 years.

### Study Treatments:

### Investigational Product (IP): Oregovomab (MAb-B43.13)

Dosage Form: Lyophilized formulation
Dose: 2 mg, over 20±5 minutes
Mode of Administration/Location: Intravenous/In clinic
Frequency: In Clinic on Day 1 of Week 1, Week 4, Week 7, Week 12, and Week 20 (see Time and Events Schedule).

### Combination Agent: Niraparib

Dosage Form: tablets
Dose: 300 mg (3×100 mg tablets)
Mode of Administration/Location: Oral/At home
Frequency: Daily to the end of week 12 for all subjects, daily administration between week 12 and week 24 will be per investigator discretion.

Comparator: Not applicable.

Placebo: Not applicable.

### Prior and Concomitant Medications:

Any medication or inactivated vaccine (including over the counter or prescription medicines, vitamins, and/or herbal supplements) deemed for supportive care and safety of the subject received at the time of enrollment or receives during the study must be recorded in the eCRF along with reason for use, dates of administration including start and end dates, and dosage information including dose and frequency.

For oregovomab, the use of pre-infusion medication and post-infusion medication is allowed to prevent or treat infusion reactions. Subjects may be pretreated with corticosteroids (such as dexamethasone), diphenhydramine, and H2 antagonists (such as cimetidine or ranitidine).

The subject must notify the Investigator about any new medications taken after the start of the study drug. All medications (other than study drug) and significant non-drug therapies (including physical therapy, herbal/natural medications and blood transfusions) administered during the study must be listed in the eCRF.

Other concomitant medication not listed above may be considered on a case-by-case basis by the investigator in consultation with the Medical Monitor if needed.

Prohibited Concomitant Medications or Medications Taken with Caution:

### For Oregovomab:

Monoamine oxidase inhibitors (MAOI) should be avoided, if possible, as they may interact with presser agents (e.g., SC epinephrine), which may cause severe hypertension. Systemic doses of immunosuppressive drugs (such as corticosteroids, methotrexate, or immune suppressive monoclonal antibodies) where the subject is treated chronically are prohibited. Subjects who have been treated with chronic immunosuppressive drugs in the past, must have been off treatment for at least 8 weeks before starting on study.

Other investigational drugs within 30 days of Week 1 Day 1 or during the study.

### Prohibited Therapies or Therapies Requiring Caution for Niraparib:

Systemic anticancer or biological therapy.
Immunotherapy.

| Humoral Immune | Humoral response to oregovomab |
|---|---|
| Characterizations | (Human Anti-Mouse Antibody (HAMA)) |
| Tumor Marker: | CA-125 |

Chemotherapy.
Investigational agents other than niraparib and oregovomab
Radiation therapy encompassing >20% of the bone marrow is prohibited within 2 weeks prior to Day 1 and during study treatment. Note: Palliative radiation therapy to a small field >1 week prior to Day 1 of study treatment may be allowed.
Any surgery that involves tumor lesions. Note: Administration of radiation therapy or surgery done that involves tumor lesions will be considered as disease progression at the time the procedure is performed.
Niraparib weakly induces Cytochrome P450 (CYP)1A2 in vitro and is a relatively poor substrate for P-glycoprotein (P-gp); therefore, investigators are advised to use caution with the substrates forCYP1A2 with a narrow therapeutic range, i.e., theophylline and tizanidine.

Prophylactic cytokines (i.e., granulocyte colony-stimulating factor [GCSF]) should not be administered in the first cycle of the study but may be administered in subsequent cycles according to current American Society of Clinical Oncology (ASCO) guideline.

### Biomarker Analyses:

### Pharmacokinetic Analyses:

Individual subject PK parameters will not be collected in this study.

### Statistical Methods:

### Sample Size Determination:

A sample size of 10 subjects is estimated to provide adequate initial descriptive assessment of safety interactions for the combination therapy and provide preliminary evidence of clinical and immune response patterns.

### Analysis Populations:

The Intent-to-treat (ITT) population will include all subjects who received the first study treatment. The ITT analysis set will serve as the primary analysis population for all efficacy endpoints and demographics.

The Per-protocol (PP) population will include all evaluable subjects who have received at least 3 doses of oregovomab and have no major protocol violations that could influence the assessment of efficacy. The PP population will be used as supportive for efficacy endpoints.

The Safety population will include all subjects who received any amount of study drug (niraparib, or oregovomab). The Safety population will be the primary analysis set for safety endpoints.

### Analysis of the Secondary Endpoint:

Progression-free survival will be calculated from the date of first study treatment to the earliest date of disease progression as determined by the local radiologists/investigators or date of death. Overall survival will be calculated from the date of first study drug treatment to the date of death.

### Safety:

Safety will be assessed by Treatment Emergent Adverse Events (TEAEs) as per CTCAE v5.0, physical examinations, laboratory assessments, and vital signs.

### Analysis Timepoints:

Data analysis will be done at the following timepoints:

Interim Analysis after at least 10 evaluable subjects have completed 12 weeks of treatment.

Primary Analysis will be performed after all subjects have completed the full 24-week treatment plus 30-day safety follow-up visit.

To be included in the interim analysis, the following criteria must be met:
Subjects must have had at least 3 doses of oregovomab.
Subjects must have available data (screening and at least one post-treatment sample collected) for biological responses (CA125/HAMA, etc.).
Subjects have completed at least two imaging assessments.

At least 10 evaluable patients will be enrolled Three groups will be analyzed:
1. All patients enrolled in study (intent to treat). DCR. HAMA, and LTFU will be reviewed.
2. Patients who took niraparib for the entire study of 24 weeks.
3. Patients who switched to alternative therapy after Week 12, with Week 12 as Baseline

Clinical Study Report (CSR) will be prepared after the last subject has completed the 30-day Safety Follow Up. The Sponsor reserves the option to complete a CSR Addendum to account for any long-term survival data, if available.

### Data Safety Monitoring Board:

No formal Data Safety Monitoring Board (DSMB) will be instituted for this study.

Instead, a Safety and Data Review Committee (SDRC), consisting of Investigators and Sponsor representatives, will be established to monitor safety during the study.

### Independent Review Committee:

No independent review committee (IRC) will be established for this study.

### Steering Committee:

No steering committee will be established for this study.

### BACKGROUND INTRODUCTION

### Introduction

Ovarian cancer represents 5% of all cancer deaths in women and is the fifth highest among all cancers and the fourth highest in the 40-57-year age subgroup. It is estimated that there will be approximately 22,530 new cases and 13,980 deaths due to ovarian cancer in the US in 2019 (ACS 2019). A woman's risk at birth of having ovarian cancer in her lifetime is approximately 1.3%, with the risk of dying of ovarian cancer almost 1.0% with little or no racial distinction. This translates to an overall death rate of 9.5 per 100,000 women per year. Ovarian cancer can be considered dangerous in that symptomology can be minimal and non-specific resulting in about 80% being diagnosed when regional or distant spread has already occurred. Approximately 70% of cases are diagnosed at ages greater than 54 years (median: 63 years old) and the median age of death due to recurrent ovarian cancer is 70 years old. The five-year survival rate for ovarian cancer is approximately 47% (ACS 2019).

Although complete responses are common (more than 50%) following initial treatment with surgery and chemotherapy (platinum and taxane regimens), within two years of cytoreductive surgery for Stage Ill, Stage IV disease, one-half of tumors recur and often there is no curative treatment. While death rates have been declining by approximately 2.2% each per year over the past ten years, there still remains considerable room for improvement in treatment to minimize recurrence rates, prolong the relapse duration and improve overall survival. Once the patient relapses, there is often no curative therapy and recurrent ovarian cancer is usually fatal.

Existing (or Available) Therapies and their Effectiveness

NCCN Guidelines Version 1.2019, suggest no single therapeutic agent should be currently recommended as the treatment of choice for recurrent ovarian carcinoma. Some regimens and agents are preferred based on expert opinion primarily for reasons of decreased toxicity and/or marginally increased effectiveness.

Platinum-based combination chemotherapy is recommended for platinum-sensitive recurrence. For subjects with platinum-sensitive disease who cannot tolerate combination therapy, the preferred single agent is carboplatin or cisplatin. Preferred combinations for platinum-sensitive disease include carboplatin/paclitaxel, carboplatin/liposomal doxorubicin, carboplatin/gemcitabine with or without bevacizumab. Although a longer platinum free interval has been associated with better outcomes, the strategy of artificially extending this interval with a non-platinum regimen does not appear to improve survival outcomes compared with the use of platinum.

Maintenance therapy is being increasingly used in the treatment of ovarian cancer. Subjects with platinum-sensitive recurrent disease may receive treatment with a combination of carboplatin and either paclitaxel or gemcitabine, plus bevacizumab, and bevacizumab can be given alone as maintenance therapy. OCEANS was a phase 3 trial that evaluated subjects with platinum-sensitive disease treated with gemcitabine and carboplatin, with and without bevacizumab, also showing improved PFS, but no difference in OS (Aghajanian et al. 2012). GOG-0213 showed that the addition of bevacizumab to standard chemotherapy, followed by maintenance therapy until progression, improved the median overall survival in subjects with platinum-sensitive recurrent ovarian cancer. Although the intention-to-treat analysis for overall survival was not significant, a sensitivity analysis based on corrected treatment-free interval stratification indicated that this strategy might be an important addition the way bevacizumab is used (Coleman et al. 2017). The AURELIA study evaluated the treatment of subjects with platinum resistant disease with single agent chemotherapy (pegylated liposomal doxorubicin, weekly paclitaxel, or topotecan), alone or with bevacizumab, showing improved PFS, but not OS (Pujade-Lauraine et al. 2014).

Alternative agents that can be chosen for maintenance are poly-ADP ribose polymerase (PARP) inhibitors, and several are approved in this setting and being increasingly used. For subjects with platinum-sensitive relapsed ovarian cancer with a partial or complete response to platinum-based chemotherapy, the PARP inhibitors niraparib, olaparib, and rucaparib are approved by the FDA (Refer to prescribing information).
Niraparib is approved for the maintenance of adult subjects with recurrent epithelial ovarian, fallopian tube, or primary peritoneal cancer who are in a complete or partial response to platinum-based chemotherapy. Niraparib is also approved for the treatment of adult subjects with advanced ovarian, fallopian tube, or primary peritoneal cancer who have been treated with three or more prior chemotherapy regimens and whose cancer is associated with homologous recombination deficiency (HRD) positive status defined by either a deleterious or suspected deleterious BRCA mutation, or genomic instability and who have progressed more than six months after response to the last platinum-based chemotherapy.
Olaparib is approved for the maintenance of adult subjects with recurrent epithelial ovarian, fallopian tube or primary peritoneal cancer, who are in a complete or partial response to platinum-based chemotherapy and for the treatment of adult subjects with deleterious or suspected deleterious germline BRCA-mutated (gBRCAm) advanced ovarian cancer who have been treated with three or more prior lines of chemotherapy, based on an FDA-approved companion diagnostic for olaparib. A further indication is for treatment of adult subjects with deleterious or suspected BRCAm advanced ovarian cancer who have been treated with three or more prior lines of chemotherapy selected for therapy based on an FDA-approved companion diagnostic for olaparib.
Rucaparib is approved for the maintenance of adult subjects with recurrent epithelial ovarian, fallopian tube, or primary peritoneal cancer who are in a complete or partial response to platinum-based chemotherapy and for the treatment of adult subjects with deleterious BRCA mutation (germline and/or somatic)-associated epithelial ovarian, fallopian tube, or primary peritoneal cancer who have been treated with two or more chemotherapies, based on an FDA-approved companion diagnostic for rucaparib.

### Description of Study Drugs

### Oregovomab

Oregovomab is an investigational monoclonal antibody that has been studied in clinical trials as an immunotherapeutic treatment (indirect immunization) of ovarian cancer, which expresses the tumor associated antigen, CA125 (MUC16). CA125 is a surface glycoprotein antigen that is expressed by most non-mucinous epithelial ovarian carcinomas. It is shed into the blood such that elevated levels in the serum can be used as a marker of ovarian cancer activity (Fendrick et al, 1993). CA125 is associated with a large molecular weight mucin-like glycoprotein complex of 200-250 kDa with a complex structure (Fendrick et al, 1993; Yin et al, 2002), and can be expressed in other inflammatory conditions of the peritoneal mucosa making the marker non-specific for effective use as a cancer screen in a general population. CA125 is most often elevated above the normal range in the presence of ovarian cancer and does provide a useful marker of disease activity when following ovarian cancer subjects longitudinally.

The active component of oregovomab (formerly OvaRex®) is the murine monoclonal antibody B43.13, an IgG1κ subclass immunoglobulin that binds with high affinity (1.16×10¹⁰/M) to CA125. Its interaction with circulating and tissue associated CA125 modifies the immune response to the tumor antigen and is the basis for treatment activity.

Oregovomab has a unique mechanism of action (MOA) based on immunological stimulation following its binding to CA125. Current evidence supports that this binding in vivo renders the target antigen CA125 more immunogenic or “neoantigen-like” through altered and enhanced antigen processing and presentation to specific T cells. This induces antigen-antibody uptake and processing using the immunoglobulin Fcγ binding, a binding pattern in the human unique to murine IgG1 resulting in cross presentation of CA125 peptides and initiation of local specific immune responses with an IFN-γ signature. These properties initiate demonstrable humoral and cellular responses in subjects with CA125-positive cancer that are otherwise in a state of relative immune tolerance to their disease. The activity has been shown to be particularly enhanced when oregovomab is given in combination with selected chemotherapy in a specific schedule in subjects with Stage III or Stage IV epithelial ovarian, tubal, or primary peritoneal cancer in the front-line setting after optimal debulking surgery with residual disease less than 1 cm.

This application of monoclonal antibody technology is different from classical active immunization to induce protective immunity (requiring the administration of the antigen as an intact molecule, molecular fragment or the antigen's genetic sequence for expression in situ) or passive immunization to directly target disease using mechanisms such as antibody-dependent cell-mediated cytotoxicity (ADCC) (requiring the administration of a large volume of specific antibody). Indirect immunization involves transient repeated exposure to a lower dose of specific antibody, avoiding gross antibody excess, and allows immune stimulatory antigen processing in the tumor microenvironment and additional systemic sites. Induced cellular immunity targeting tumor antigen is believed to be the primary mechanism of action. The principle has been explored in multiple antigen systems, and for ovarian cancer specifically targeting CA125.

Indirect immunization with oregovomab is an approach to increase the immune recognition of CA125 within the subject who has cancer and to initiate an anti-tumor immune response. Previous studies have demonstrated that such immune initiation alone in the setting of immune suppressive malignancy is insufficient to alter the clinical course of disease (Berek et al. 2004, Berek et al. 2009), however if additional attention is focused on the immune modulatory properties of co-administered cancer therapies in a schedule dependent fashion, an impact on subject outcomes is possible.

In 2009, Braly published a randomized phase 2 study of subjects with frontline Stage Ill or Stage IV disease post-surgery in which oregovomab, 2 mg per infusion, was simultaneously administered with standard chemotherapy, carboplatin and paclitaxel, in one group of subjects and a week after chemotherapy in the other. The study showed that the arm subjected to simultaneous immuno-chemotherapy developed a better immune response using several orthogonal metrics (contrary to what was previously thought considering the immunosuppressive effects of chemotherapy) (Braly et al. 2009). The schedule dependency was detected in assays of humoral response to administered antibody and in prototype assays of cellular immunity to CA125 using an enzyme-linked immunosorbent spot (ELISPOT) assay platform. An early humoral response measured after 6 weeks was detected in 75% of patients receiving the preferred schedule of chemo-immunotherapy relative to 35% in the alternate delayed schedule of chemoimmunotherapy (Braly et al. 2009). These data supported further clinical exploration in a randomized Phase 2 study QPT-ORE-002. Final results were published as (Brewer et al. 2020 and Battaglia et al. (translational results) (CII 2020.), and are described below, showing clinically significant improvement in PFS and OS with little added toxicity when oregovomab is added to a standard regimen of chemotherapy, paclitaxel/carboplatin, after optimal debulking surgery in subjects with Stage III and IV epithelial ovarian, fallopian tube, or primary peritoneal cancer.

These phase 2 results of QPT-ORE-002 support the concept that the addition of oregovomab in a schedule dependent manner to a chemotherapy regimen such as paclitaxel/carboplatin, provides a meaningfully improved magnitude of efficacy via a unique MOA, with minimal additional toxicity (a highly favorable safety profile). It also reinforces the improved effectiveness paradigm of supplementing oregovomab with a non-chemotherapy drug such as niraparib, known to be effective as a maintenance therapy in adults with recurrent epithelial ovarian, fallopian tube, or primary peritoneal cancer who are in partial response to platinum-based chemotherapy.

### Niraparib

Niraparib is a potent, orally active PARP1 and PARP2 inhibitor being developed as a treatment and maintenance therapy for subjects with tumors that harbor defects in the homologous recombination DNA repair pathway or that are driven by PARP-mediated transcription factors and is also being evaluated in subjects who are HRD negative and non-BRCA-mutated.

Poly (ADP-ribose) polymerase (PARP), PARPs 1 and 2, are zinc-finger deoxyribonucleic acid (DNA)-binding enzymes that play a crucial role in DNA repair (Fong et al. 2009). Upon formation of DNA breaks, PARP binds at the end of broken DNA strands, a process that activates its enzymatic activity.

Activated PARP catalyzes the addition of long polymers of adenosine diphosphate (ADP)-ribose onto PARP and several other proteins associated with chromatin, including histones and various DNA repair proteins (De Lorenzo et al. 2013, Jones et al. 2015). This results in chromatin relaxation, fast recruitment of DNA repair proteins, and efficient repair of DNA breaks. In this manner, PARP plays a key role in sensing DNA damage and converting it into intracellular signals that activate the base excision repair (BER) and single-strand break repair pathways.

Normal cells repair up to 10,000 DNA defects daily, and single-strand breaks are the most common form of DNA damage. Cells that are unable to repair this burden of DNA damage, such as those with defects in the homologous recombination or BER pathways, are at risk for accumulating multiple lesions that will ultimately trigger apoptosis. They enter the S phase (DNA replication) of the cell cycle with unrepaired single- and double-strand breaks. Pre-existing single-strand breaks are converted to double-strand breaks as the replication machinery passes. Accumulated double-strand breaks present during S phase are repaired by homologous recombination. Homologous recombination is the preferred repair pathway because it is associated with a much lower error rate than other forms of repair.

Cells that are unable to perform DNA repair via homologous recombination (e.g., due to inactivation of genes required for homologous recombination, such as breast cancer [BRCA1] or breast cancer 2 [BRCA2]-mutated cells), are at risk for accumulating multiple lesions that will ultimately trigger apoptosis. These cells accumulate stalled replication forks during S phase and are more likely to use the error-prone non-homologous end joining (NHEJ) or alternative (alt)-NHEJ pathways to repair double-strand breaks in DNA. Accumulation of errors in DNA by NHEJ contributes to mutation burden that promotes the development of cancer. Over time, the buildup of excessive DNA errors in combination with the inability to complete S phase because of stalled replication forks, contributes to cell death (De Lorenzo et al. 2013, Jones et al. 2015). In this way, treatment with PARP inhibitors could represent a novel opportunity to selectively kill cancer cells. For example, a tumor arising in a patient with a germline BRCA mutation (gBRCAmut) has a defective homologous recombination DNA repair pathway and would be increasingly dependent on NHEJ, alt-NHEJ, and BER for maintenance of genomic integrity. PARP inhibitors block alt-NHEJ and BER, forcing tumors with BRCA deficiencies to use the error-prone NHEJ to fix double-strand breaks (Fong et al. 2013). Non-BRCA deficiencies in homologous recombination DNA repair genes could also enhance tumor cell sensitivity to PARP inhibitors (Gelmon et al. 2011). The rationale for anti-cancer activity in a subset of non-gBRCAmut tumors is that they share distinctive DNA repair defects with gBRCAmut carriers, a phenomenon broadly described as “BRCAness” (Turner et al. 2004). DNA repair defects can be caused by germline or somatic alterations to the homologous recombination DNA repair pathway. In a recent analysis of approximately 500 high-grade serous ovarian adenocarcinoma tumors, approximately 50% contained HRD (CGA 2011). A subset of these tumors had biologically plausible molecular alterations that may make them sensitive to PARP inhibition by niraparib. A similar analysis of triple-negative breast cancer indicates that 43% to 44% of these subjects have tumors with HRD (Timms et al. 2014). Homologous recombination is a complex pathway, and several genes other than BRCA1 and BRCA2 are required either to sense or repair DNA double-strand breaks via the homologous recombination pathway. Therefore, PARP inhibitors are also selectively cytotoxic for cancer cells with deficiencies in DNA repair proteins other than BRCA1 and BRCA2 (Turner et al. 2011). Recent clinical studies have shown PARP inhibitors to be active in breast and ovarian cancer, and potentially other tumor types (Pilié P G, et. al.) Clinical anti-cancer activity with PARP inhibitors has been seen in both subjects with gBRCAmut and without gBRCAmut; however, activity is more robust in subjects with the germline mutation. (Kummar et al. 2012)

In summary, treatment with PARP1/2 inhibitors represents a novel opportunity to selectively kill a subset of cancer cell types by exploiting their deficiencies in DNA repair. Human cancers exhibit genomic instability and an increased mutation rate due to underlying defects in DNA repair. These deficiencies render cancer cells more dependent on the remaining DNA repair pathways, and targeting these pathways is expected to have a much greater impact on the survival of the tumor cells than that of normal cells. The rationale for combining niraparib with oregovomab is described in more detail below.

### Pre-Clinical Experience of Oregovomab

### Nonclinical Pharmacology

Oregovomab MAb-B43.13 does not show any significant cross-reactivity with selected normal human organs and tissues but does show selective binding to adenocarcinomas expressing the CA125 antigen. MAb-B43.13 does not have antibody-dependent cell cytotoxicity (ADCC) or complement-dependent cytotoxicity (CDC) properties either in vitro or in vivo. MAb-B43.13 induces a strong humoral response in normal rats, normal rabbits and tumor-bearing mice. Cellular responses were evident in the tumor-bearing mice. Biological activity was demonstrated in tumor-bearing mice with both an increase in survival and decrease in tumor size evident after treatment with oregovomab MAb-B43.13 compared to controls.

For more detailed information, please refer to the Oregovomab MAb-B43.13 IB.

### Pharmacokinetics in Animals

In both normal and tumor-bearing mice, the distribution of 99mTc-labeled MAb B43.13 demonstrated a lack of unusual accumulation in normal organs and tissues and a progressive elimination of the tracer over time. Tumor-bearing animals showed a preferential accumulation of the compound in CA-125-expressing tumor xenografts compared to an isotype-matched control antibody.

For more detailed information, please refer to the Oregovomab MAb-B43.13 IB.

### Toxicology

Acute toxicity studies in male and female rats and female rabbits demonstrated a lack of significant acute toxicity in both species at 100 times the estimated human dose equivalent (2 mg/60 kg body weight). Similar findings were obtained from a sub-acute toxicity study in female rabbits at 100 times the human dose weight equivalent.

For more detailed information, please refer to the Oregovomab MAb-B43.13 IB.

### Pre-Clinical Experience of Niraparib

### Nonclinical Pharmacology

Nonclinical data on niraparib are discussed in detail in the niraparib IB. Briefly, in pre-clinical models, niraparib has been observed to inhibit normal DNA repair mechanisms and induce synthetic lethality when administered to cells with HRD. In a BRCA1-mutant xenograft study, niraparib dosed orally caused tumor regression, which was mirrored by a >90% reduction in tumor weight compared with control. In a BRCA2-mutant xenograft study, niraparib-dosed mice showed 55% to 60% growth inhibition, both by tumor volume and weight.

Niraparib displayed strong antitumor activity in in vivo studies with BRCA1-mutant breast cancer (MDA-MB-436), BRCA2-mutant pancreatic cancer (CAPAN-1), and with patient-derived Ewing sarcoma mouse models. Utilizing patient-derived ovarian and breast cancer xenograft models, niraparib demonstrated response in both BRCAmut and BRCA wild-type tumors.

### Clinical Experience

This is a first-in-human study of the combination of oregovomab and niraparib, so no data on the combination exists. Information will be provided from experience with each drug separately.

### Clinical Safety

### Oregovomab

A total of 963 subjects with ovarian cancer were studied in 10 clinical studies conducted from 1997 through 2008 (649 subjects treated with oregovomab and 314 subjects treated with placebo). In addition, QPT-ORE-002, conducted from 2011 through 2018, studied an additional 97 subjects with ovarian cancer (safety population of 94: 46 subjects treated with oregovomab+paclitaxel/carboplatin and 48 subjects treated with placebo+paclitaxel/carboplatin). Collectively, oregovomab or placebo has been administered at a dose of 2 mg/50 mL as a 20-minute intravenous infusion to a total of 1057 subjects with ovarian cancer. The number of administered doses per subject has ranged from one (1) to twenty (20).

For more information on clinical safety, please refer to the Oregovomab MAb-B43.13 IB.

### Niraparib

The primary data to support the safety of treatment with niraparib are derived from the NOVA study in which a total of 546 subjects received study treatment.

All 367 subjects who received niraparib and 171 (96%) of 179 subjects who received placebo experienced at least 1 treatment-emergent adverse event (TEAE). The high rate of TEAEs in the placebo group indicates the burden of prior chemotherapy and the subject's underlying ovarian cancer. Review of the data across study cohorts for TEAE incidence showed that, in general, the results were similar in the gBRCAmut and non-gBRCAmut cohorts. In the overall safety population, for the niraparib versus placebo treatment arms, the incidences of Grade 3 or 4 TEAEs (74% vs. 23%), serious adverse events (SAEs) (30% vs. 15%), TEAEs leading to treatment interruption (67% vs. 15%), TEAEs leading to dose reduction (69% vs. 5%), and TEAEs leading to treatment discontinuation (15% vs. 2%) were higher for niraparib than for placebo. There were no on-treatment deaths reported.

The most commonly observed non-hematologic TEAEs (all grades) in niraparib-treated compared with placebo-treated subjects were nausea (74% vs. 35%), fatigue (46% vs. 32%), constipation (40% vs. 20%), and vomiting (34% vs. 16%). The majority of the non-hematological TEAEs were mild to moderate in severity. The most commonly observed hematologic TEAEs (all grades) of niraparib were anemia (49%), thrombocytopenia (46%), decreased platelet count (20%), and neutropenia (18%). Although Grade 3 or 4 hematologic laboratory AEs were common at the initiation of study treatment, no severe clinical sequelae were observed, and relatively few subjects discontinued study treatment due to these AEs. Dose adjustment based on individual tolerability during the first 3 cycles substantially reduced the incidence of these AEs beyond Cycle 3, indicating the overall effectiveness of the approach to dose modification. These TEAEs can be monitored routinely using standard assessments of hematological laboratory parameters, as is routine for subjects with ovarian cancer receiving anticancer therapies. In the NOVA study, niraparib dose adjustment tended to occur early with most subjects reaching their individual adjusted dose level at the end of Month 3 (i.e., Cycle 3) of treatment.

Myelodysplastic syndrome (MDS) and acute myeloid leukemia (AML) have been observed in subjects receiving treatment with olaparib, a PARP inhibitor; given the common mechanism of action, MDS and AML therefore represent a potential risk to subjects receiving niraparib. In the Phase 3 NOVA study, the incidence of MDS/AML in subjects who received niraparib (5 of 367; 1.4%) was similar to its incidence in subjects who received placebo (2 of 179; 1.1%). Guidance on monitoring subjects for new AEs of MDS/AML and the follow-up of subjects with suspected MDS/AML is provided.

Study PR-30-5011-C1 (NOVA corrected QT interval [QTc] sub study; n=26) is an open-label evaluation of the effects of niraparib on QTc measurements in subjects with histologically diagnosed ovarian cancer, fallopian tube cancer, or primary peritoneal cancer. There were no reports of clinically significant abnormal electrocardiogram (ECG) changes, including QTc interval prolongation, attributed to niraparib. Administration of niraparib at the therapeutic dose did not prolong the QT interval. There was no correlation between the exposure level (ie, plasma concentration) of niraparib and QTc changes (ie, change in corrected QT interval calculated using Fridericia's formula [ΔQTcF]).

For more information on clinical safety, please refer to the Niraparib IB.

### Niraparib Baseline Platelet Count and Weight as Predictors of Thrombocytopenia

An analysis was conducted using the data collected in ENGOT-OV16/NOVA and the initial phase I study, PN001. This analysis determined that baseline platelets had an impact on platelet nadir; lower baseline platelets (<180×10⁹/L) were associated with an increased frequency of thrombocytopenia Grade ≥1 (76%) or Grade ≥3 (45%) compared to subjects with higher baseline platelet counts. Further, an exploratory analysis of clinical data versus baseline body weight from ENGOT-OV16/NOVA was conducted. For this analysis, the weight categories were based on quartiles with the lowest quartile (subjects with a body weight less than 58 kg at baseline) compared to the highest quartile (subjects with a body weight greater than or equal to 77 kg at baseline). While TEAEs occurred in most subjects regardless of body weight, Grade ≥3 TEAEs, SAEs, and TEAEs leading to dose modification or treatment discontinuation occurred more commonly in the weight <58 kg cohort than in the ≥77 kg cohort. In the cohort of subjects with a body weight <58 kg, approximately 80% of subjects had a dose reduction compared to 59% of subjects with a weight greater than or equal to 77 kg. Treatment discontinuations were increased in the subjects with lower body weight (24%) compared to subjects in the highest quartile (10%).

The potential relationship between body weight and TEAEs was further explored in an analysis to evaluate the correlation of grade 3 or 4 thrombocytopenia and baseline body weight. The lowest platelet count in the first 30 days was plotted versus baseline body weight to determine if low body weight identified a subgroup of subjects with higher levels of thrombocytopenia during Cycle 1. In the first 30 days of treatment, a baseline body weight ≥77 kg is associated with a lower incidence of grade 3 or 4 thrombocytopenia (14%) relative to the group with body weight <58 kg (43%).

Finally, a classification tree approach was used to refine the best cut-off points for predicting the likelihood of a subject developing ≥Grade 3 thrombocytopenia within 30 days after the first dose of niraparib (Berek et. al, 2018). The results of the model show that the subgroup of subjects with a baseline body weight <77 kg or baseline platelet count <150,000 μL had a grade 3/4 thrombocytopenia rate in the first 30 days of 35.4% compared to 11.5% in the group of subjects with a body weight >77 kg and a platelet count >150,000 μL. Further, the average daily dose was 258 mg through the first two cycles for subjects with a body weight >77 kg and platelet count >150,000 μL and was only 206 mg for subjects with body weight <77 kg or platelet count <150,000 μL. Thus, the actual delivered dose approximated a starting dose of 200 mg despite the intended delivery of a starting dose of 300 mg. Also, retrospective analyses from NOVA demonstrated that the reduced dose did not compromise efficacy. This was recently shown prospectively in the phase 3 PRIMA/ENGOT-OV26/GOG-3012 study which evaluated maintenance therapy with niraparib vs placebo after response to platinum-based chemotherapy in subjects with newly diagnosed advanced high-grade serous or endometrioid ovarian, primary peritoneal, or fallopian tube cancer (unpublished data).”. These observations are to be confirmed in the present study with the inclusion of study treatment dosed at 200 mg (2 tablets of niraparib) in subjects whose baseline weight is <77 kg and baseline platelet count is <150,000 μL.

### Clinical Efficacy

### Oregovomab

The final results for QPT-ORE-002 mentioned above are relevant to the concept that the activity of oregovomab is enhanced when added to other anticancer agents, and were as follows: the phase 2 prospective, randomized study (QPT-ORE-002) of 97 subjects accrued at 13 sites in the US and Italy. Subjects received either paclitaxel-carboplatin-oregovomab or paclitaxel-carboplatin alone. A total of 97 subjects, with previously untreated, advanced epithelial ovarian, adnexal, or primary peritoneal cancer, Stage III and Stage IV, were treated after undergoing optimal debulking surgery (residual disease <1 cm in diameter) in 10 centers (Italy and US) and followed for 3 years after treatment.

Final results revealed that subjects treated with oregovomab+paclitaxel/carboplatin had a clinically significant improved PFS (median 41.8 months) compared to paclitaxel/carboplatin alone (median 12.2 months) by the K-M method (p=0.0027) and a HR=0.44 by multivariate Cox PH method (p=0.0029). The subjects treated with oregovomab+paclitaxel/carboplatin had a clinically significant improved OS (median NE) compared to paclitaxel/carboplatin alone (median 43.2 months) by the K-M method (p=0.0042) and a HR=0.34 by the multivariate Cox PH method (p=0.0077), as well as a clinically significant improved time to clinical progression (TTCP) (median 43.1 months) compared to paclitaxel/carboplatin (median 13.6 months) by the K-M method (p=0.0014) and a HR=0.40 by the multivariate Cox PH method (p=0.0019).

For more information for clinical efficacy, please refer to the Oregovomab MAb-B43.13 IB.

### Niraparib Phase 3 Study of Niraparib Monotherapy in Platinum-Sensitive, Recurrent Ovarian Cancer

Recent results have been published demonstrating that niraparib is more efficacious than placebo as maintenance therapy for subjects with platinum-sensitive, recurrent ovarian cancer (NOVA study), and niraparib has activity as a monotherapy in the fourth or later line of treatment (QUADRA study), which are both relevant as background for this proposed oregovomab+niraparib study.

### NOVA Study

In the randomized, double-blind, Phase 3 NOVA trial (Niraparib Maintenance Therapy in Platinum-Sensitive, Recurrent Ovarian Cancer), a total of 553 subjects were randomized at 107 centers worldwide (Mirza, 2016). The purpose of the study was to evaluate the efficacy of niraparib versus placebo as maintenance for subjects with platinum-sensitive, recurrent ovarian cancer. Subjects were categorized according to the presence or absence of a gBRCAmut (gBRCA cohort and non-gBRCA cohort) within their tumors and the type of non-gBRCAmut and were randomly assigned in a 2:1 ratio to receive niraparib (300 mg) or placebo once daily (QD). The primary end point was PFS. The study enrolled 203 subjects in the gBRCAmut cohort and 350 subjects in the non-gBRCAmut cohort. Among the 350 subjects in the non-gBRCAmut cohort, 162 had tumors that were identified as HRD positive (HRDpos), and 134 had tumors that were HRD negative (HRDneg). HRD status was not determined for 54 subjects.

Demographic and baseline characteristics were well balanced. Table 1 below shows the results for the PFS primary endpoint for each of the 3 primary efficacy populations (i.e., gBRCAmut cohort, HRDpos cohort, and overall non-gBRCAmut cohort). In addition, median PFS in subjects with HRDneg tumors was 6.9 months (95% confidence interval [CI]: 5.6, 9.6) in the niraparib arm, versus 3.8 months (95% CI: 3.7, 5.6) in the placebo arm, with a HR of 0.58 (95% CI: 0.361, 0.922) (p=0.0226).

TABLE 1
Progression-Free Survival in Ovarian Cancer Subjects in NOVA

| | | | Non-gBRCAmut Cohort | | HRDpos (Within | |
|---|---|---|---|---|---|---|
| | | | (Regardless of HRD | | non-gBRCAmut | |
| | gBRCAmut Cohort | | Status) | | Cohort) | |
| | Niraparib | Placebo | Niraparib | Placebo | Niraparib | Placebo |
| | (n = 138) | (n = 65) | (n = 234) | (n = 116) | (n = 106) | (n = 56) |
| Median PFS (95% CI) ⁽¹⁾ | 21.0 | 5.5 | 9.3 | 3.9 | 12.9 | 3.8 |
| | (12.9, NE) | (3.8, 7.2) | (7.2, 11.2) | (3.7, 5.5) | (8.1, 15.9) | (3.5, 5.7) |
| p-value⁽²⁾ | <0.0001 | | <0.0001 | | <0.0001 | |
| HR | 0.27 | | 0.45 | | 0.38 | |
| (niraparib:placebo) | (0.173, 0.410) | | (0.338, 0.607) | | (0.243, 0.586) | |
| (95% CI)⁽³⁾ | | | | | | |

Source: PR-30-5011-C (NOVA main) CSR
^{a} PFS is defined as the time in months from the date of randomization to progression or death.
^{b} Based on stratified log-rank test using randomization stratification factors.
^{c} Based on the stratified Cox proportional hazards model using randomization stratification factors.

### QUADRA Study

Clinical activity of PARP inhibitors (PARPi) as a monotherapy has also been reported in the recurrent ovarian cancer treatment setting in the QUADRA (Moore et al, 2019) trial (NCT02354586). The purpose of this latest study was to investigate late-line treatment options for subjects with ovarian cancer where effective options are few, with the proportion of subjects achieving an overall response typically less than 10%, and median overall survival after third-line therapy of 5-9 months. In this study, the activity of niraparib monotherapy as the fourth or later line of therapy was investigated.

There were 463 subjects treated with single agent niraparib in subjects with (HRD)-positive tumors (including subjects with and without BRCA mutations) sensitive to their last platinum-based therapy, and who had received three or four previous anticancer therapy regimens. Subjects with prior exposure to PARP inhibitors were excluded. Subjects without BRCA mutations must have progressed at least six months after the last dose of platinum-based therapy. HRD-positive status was determined using the Myriad myChoice CDx as either tumor BRCA mutated (tBRCAm) (n=63) and/or a genomic instability score (GIS) 42 (n=35). All subjects received 300 mg of niraparib once daily until disease progression or unacceptable toxicity.

The primary efficacy outcome measures were objective response rate (ORR) and duration of response (DOR), as assessed by the investigator using RECIST v1.1. Of the 98 subjects in the HRD-positive cohort, ORR was 24% (95% CI: 16, 34). All were partial responses. Estimated median DOR was 8.3 months (95% CI: 6.5, not estimable). For subjects with tBRCAm ovarian cancer, ORR was 39% (7/18; 95% CI: 17, 64) in subjects with platinum-sensitive disease, 29% (6/21; 95% CI: 11, 52) in those with platinum-resistant disease, and 19% (3/16; 95% CI: 4, 46) in subjects with platinum-refractory disease. Thirteen (28%) of 47 subjects in the primary efficacy population achieved an overall response (95% CI 15.6-42.6; one-sided p=0.00053). This was a clinically relevant activity of niraparib among women with heavily pretreated ovarian cancer, especially in subjects with HRD-positive platinum-sensitive disease, which includes not only subjects with a BRCA mutation but also a population with BRCA wild-type disease. No new safety signals were identified.

For more information for clinical efficacy, please refer to the niraparib IB.

### Clinical Pharmacokinetics

### Oregovomab

Based on data from study OVA-Gy-01, the apparent terminal half-life (t_{1/2z}) of oregovomab on first infusion is around 33 hours and the plasma clearance around 110 mL/minute for a mean residence time (MRT) of about 45 hours. Slightly less than 50% of the dose of the radiotracer is eliminated in the urine, presumptively in the form of free ^{99m}Technetium. Clearance is accelerated on repeat administration due to generation of early humoral responses directed to this xenotypic immunogenic antibody.

For more information, please refer to the Oregovomab MAb-B43.13 IB.

### Niraparib

For more information, please refer to the Niraparib IB.

### Study Rationale

This proposed study is a single arm, open label Phase 1b/2 evaluation of the combination of oregovomab (an indirect immunizing monoclonal antibody specific for the ovarian cancer antigen CA125) and niraparib (an orally administered Poly ADP-ribose polymerase enzyme inhibitor) in subjects with platinum sensitive ovarian, fallopian tube and primary peritoneal cancers. The combination will be evaluated as a combinatorial immune priming strategy.

Both agents, oregovomab and niraparib, have previously been used in combination with other anti-cancer treatments and demonstrated bioactivity in advanced ovarian cancer. However, they have not previously been combined with each other. Although somewhat marrow suppressive, PARP inhibition has been demonstrated to be compatible with immunotherapy (Konstantinopoulos et al. 2019). Resultant dsDNA fragments from treatment with niraparib should induce some level of immunogenic tumor cell death. The dsDNA fragments have been shown to activate the STING (STimulator of INterferon Genes) sensing pathway of innate immunity resulting in an enhanced IFN-γ inflammatory signature. Activating STING could also potentially reignite immunologically inert tumors as has been shown in mouse models in other tumor types (Jing et al. 2017). The data demonstrated that pancreatic cancer progression is potently inhibited by a STING agonist, which reignited immunologically cold pancreatic tumors to promote trafficking and activation of tumor-killing T cells. In the case of the combination of oregovomab with niraparib in ovarian cancer, hypothetically, T cells which have been immune sensitized by oregovomab may be further activated and trafficked into tumor in the presence of niraparib mediated induction of STING.

This proposed study will primarily assess the immunological and clinical activity of combining oregovomab with niraparib. It will also be a preliminary investigation of the impact on safety of indirect immunization with oregovomab combined with the immune stimulatory activity of PARP inhibition. The population of subjects with platinum sensitive recurrent disease, defined as ovarian cancer that comes back 6 or more months after platinum-based treatment, in the second to fourth line of therapy, provides a reasonably convenient window after the most recent relapse to determine potential interactions and anti-tumor activity of combination oregovomab and niraparib. Study subjects must be deemed to be clinically appropriate, in the judgment of the clinical investigator, to receive a 12-week delay before initiation of the next-line cancer therapy while they receive the experimental immune modifying treatment with oregovomab and niraparib under this protocol.

Since clinical response to the oregovomab and niraparib combination has not yet been investigated, all subjects will be treated for 12 weeks with oregovomab and niraparib therapy, then assessed for early humoral activity and DCR at 12 weeks. If there is a DCR in 4 out 10 patients (defined here as SD, PR, CR), 12 more weeks of oral niraparib should be given, unless the investigator determines that the subject should be switched to the alternative next line of cancer treatment. In case of progression at Week 12 or earlier, the subject should stop receiving niraparib and be switched immediately to an alternative cancer treatment. All subjects, regardless of choice of therapy after the first 12 weeks of oregovomab and niraparib, will be treated with one additional monotherapy dose of oregovomab at week 20. Clinical response will be assessed again at week 24.

In addition to its potential for response, there is a possibility that responses to subsequent cancer therapy may also be enhanced by the long-standing consequences of the interaction between oregovomab immune stimulation, and PARP mediated anti-tumor effects and its recently recognized ability to boost the immune system. Data (progression free and overall survival status) obtained from long term follow up may provide additional preliminary information supportive of a promising combinatorial sequence for further investigation not only of improved survival in response to the oregovomab+niraparib combination, but also its potential impact on altering the immune environment leading to more effective subsequent therapies.

The dose to be tested in this study for each component have been selected previously through human clinical study. Establishing DCR and observing for early humoral activity of the combination in this study will provide justification for further assessment in the frontline, as well as other recurrent disease settings.

In summary, this study will primarily assess potential of the drug combination to induce humoral activity and DCR on its own, and/or to enhance response and survival to subsequent therapy and ensure safety of the combination.

### Benefit/Risk Assessment

The possibility that combination with niraparib may augment oregovomab's efficacy, but with minimal addition of toxicity, needs to be established. This combination could represent a potential advance in the management of platinum-sensitive ovarian cancer over available therapies, whereby the addition of oregovomab in a schedule dependent manner to niraparib, may provide a meaningfully improved magnitude of efficacy for clinically significant endpoints (ORR, survival) via a unique MOA, with a favorable safety profile. Furthermore, there is a possibility that improved efficacy to subsequent cancer therapy may be enhanced by the interaction between oregovomab immune stimulation, and PARP mediated anti-tumor effects.

### Justification for Dose of Oregovomab

The fixed dose of oregovomab at 2 mg given in a slow 20-minute (+/−5 min) infusion is appropriate for indirect immunization and induction of immunity to a self-antigen using a low dose antibody. This dose of oregovomab is in the optimal range to induce cellular immunity which diminishes at higher doses of the antibody. Early imaging studies of radio labeled oregovomab (Mab-43.13) at the 2 mg dose level were intended to optimize signal-to-noise and limit anti-mouse antibody interactions on repeat injection. This dose was retained in numerous subsequent therapeutic clinical trials due to observed improved clinical outcome findings and immunological responses. In addition, multiple studies with oregovomab, at 2 mg given on various schedules, have provided support for the current dose (Braly et al. 2009; Brewer et al. 2020).

### Justification for Dose of Niraparib

The 300 mg dose of niraparib is the optimal pharmacologic dose approved clinically for use in ovarian cancer, balancing enzyme inhibition relative to toxicity. This dose of niraparib is associated with toxicities not seen with oregovomab, most notably a bone marrow suppression (neutropenia, thrombocytopenia, and anemia) in 20-40% of subjects, and hypertension/hypertensive crisis seen in 9% of subjects under treatment for a period of up to two years. Although somewhat marrow suppressive, PARP inhibition has been demonstrated to be compatible with immunotherapy as reported in an initial report at ASCO 2017 (Friedlander, 2017) and which has also been seen in unpublished studies (Tesaro, data on file).

Recently, the management of marrow toxicity has been simplified by reducing the dose from 300 mg to 200 mg orally per day based on either baseline body weight and/or platelet count, which reduces adverse effect on platelet counts, but maintains clinical activity.

This dose reduction was based on the ENGOT-OV16/NOVA and PN001 studies, and a model classification tree approach, which showed that subjects with a baseline body weight <77 kg and/or baseline platelet count <150,000 μL had approximately a 30% less grade 3/4 thrombocytopenia rate in the first 30 days of treatment. Further, the actual delivered dose in these subjects approximated a starting dose of 200 mg despite the intended delivery of a starting dose of 300 mg. In this present study, subjects will be treated with niraparib at 300 mg (3 tablets of niraparib). Subjects whose baseline weight is <77 kg or baseline platelet count is <150,000 μL, the daily dose of niraparib will be 200 mg (2×100 mg tablets of niraparib).

### Objectives & Endpoints

TABLE 2
Objectives and Related Endpoints

| | |
|---|---|
| Objectives | Endpoints |

Primary

| | |
|---|---|
| Disease Control Rate (DCR) | Per RECIST v1.1 as determined by the |
| To evaluate anti-tumor activity | investigator: |
| of oregovomab added to | DCR is defined as the proportion of |
| niraparib at 12 and 24 weeks | subjects with complete response (CR), |
| in subjects with CA125- | partial response (PR), and stable disease |
| associated recurrent epithelial | (SD) at Weeks 12 and 24. |
| adenocarcinoma of ovarian, | Duration of Response, defined as the |
| fallopian tube or peritoneal | time measurement criteria are met for |
| origin. | SD, CR or PR until the first date that |
| | recurrence or PD is objectively |
| | documented. |

Secondary

| | |
|---|---|
| Safety and Tolerability | Frequency/severity of vital signs |
| To establish the preliminary | measurements, physical examination |
| safety and tolerability of | findings, changes in clinical laboratory |
| oregovomab added to niraparib | parameters (hematology, biochemistry, |
| in subjects with CA125- | urinalysis), and incidence of adverse |
| associated recurrent epithelial | events (AEs), serious AEs (SAEs), |
| adenocarcinoma of ovarian, | deaths, and AEs leading to |
| fallopian tube or peritoneal | discontinuation of treatment. |
| origin. | |
| Humoral immune responses | Antibody to oregovomab as measured |
| | by HAMA |
| | Early humoral response measured at |
| | Week 7 (Day 43) relative to baseline is |
| | special interest and is evaluated serially |
| | through the treatment phase of the |
| | study. |

Exploratory

| | |
|---|---|
| To explore the effect of | Progression-Free Survival (PFS), |
| oregovomab added to niraparib | defined as date of first dose of study |
| with respect to initial | treatment to the date of event defined as |
| progression, response rate, | the first documented progression as per |
| time to subsequent therapies, | RECIST v1.1 as determined by the |
| time to next progression and | investigator or death due to any cause. |
| death. | Overall Response Rate (ORR) to |
| | second line therapy-initiated post Week |
| | 12 measured at Week 24 relative their |
| | Week 12 assessment (new baseline) |
| | Overall Survival (OS), defined as da |
| | date of first dose of study treatment to |
| | date of death due to any cause. |
| | Time to First Subsequent Therapy |
| | (TFST), defined as time from the date |
| | of first dose of study treatment to |
| | second anti-cancer therapy start date |
| | study treatment completion, |
| | following treatment exit [EOT] or |
| | death. |

Potential Immunological Responses/Biomarkers

| | |
|---|---|
| Genetic Status | Homologous Recombination |
| | Deficiency (HRD) status (patient |
| | history) |
| | BRCA 1 and BRCA2 (germline and |
| | somatic) (patient history) |

### Study Design

### Overall Design

A Phase 1 b/2 single arm open label study to evaluate the combination of oregovomab and niraparib as a combinatorial immune priming strategy in subjects with platinum sensitive recurrent ovarian cancer.

Approximately 15 subjects will be screened to enroll approximately 10 evaluable subjects. The Sponsor may, after a review of the available efficacy and safety data, amend the protocol to enroll up to 30 additional subjects for further evaluation of efficacy in this study. The decision to amend the protocol to enroll additional subjects will be based on observation of early humoral response in 5 out of 10 evaluable patients at Week 7 and/or a Disease Control Rate (DCR) of 40%, defined as CR, PR or SD in 4 out of 10 evaluable patients at Week 12 disease assessment. The study will include a:

### Screening Period (Up to 28 Days Prior to the Start of Study Treatment)

After signing informed consent, subjects will be screened for eligibility during the period of 28 days immediately prior to starting study drug on Week 1 Day 1 visit. Once all eligibility criteria are met, the subjects will be enrolled and initiate therapy.

### Treatment Period (About 24 Weeks):

Once all eligibility criteria are rechecked and met, the subject will be given study treatment.

All subjects will receive the combination of niraparib and oregovomab as follows:
Niraparib (300 mg) will be administered orally once daily starting at the first day of treatment (Day 1 Week 1). The daily dose of niraparib will be (3×100 mg tablets) taken orally from Day 1 Week 1 to the end of Week 12. Subjects whose baseline weight is <77 kg or baseline platelet count is <150,000 μL, the daily dose of niraparib will be 200 mg (2×100 mg tablets of niraparib.
   The drug should be taken at home, approximately at the same time each day. Bedtime administration may be a potential method for managing nausea.
   Each tablet should be swallowed whole.
   May be taken with or without food.
   In the case of a missed dose, subjects should take their next dose at its regularly scheduled time.
   If a subject vomits or misses a dose, an additional dose should NOT be taken.
   Subjects must bring their pill bottles to every clinic visit for clinic staff to perform drug accountability.
Oregovomab (2 mg) will be administered via IV infusion in 50 mL of normal saline over 20 minutes±5 minutes during clinic visits at Day 1 of Week 1, Week 4, Week 7, Week 12, and Week 20.

On clinic days, subjects will take niraparib 1st, then 1-2 hours later, oregovomab will be administered; this does not apply if niraparib is taken at bedtime. Subjects will be observed for 1-2 hours after the end of the oregovomab infusion.

All clinical, safety and immunological evaluations will be conducted according to a defined schedule during the treatment period and the survival follow-up period (see Appendix A). The treatment period is defined as time from the beginning of first study treatment to treatment period exit. Treatment period exit is defined as Week 24 or early termination.
Subjects who have progressive disease or unknown [PD/UNK] per RECIST v1.1, at Week 12, may discontinue niraparib and be switched to alternative treatment of Physician's choice.
Subjects who have stable disease [SD], partial response [PR], or complete response [CR], as per RECIST v1.1, at Week 12, may continue to take niraparib or may switch to alternative treatment of physician's choice.
All subjects, regardless of if they stay on niraparib or not, are required to take the 5th dose of oregovomab at Week 20 visit.

Imaging studies for disease assessment will be done at baseline, Week 12, and Week 24 visits using RECIST v1.1. Any subsequent imaging will be done according to institutional guidelines. Subjects will be treated until treatment exit at Week 24, discontinuation due to disease progression, lost to follow-up, withdrawal of consent, treatment with another anti-cancer drug, or death.

During the treatment period, subjects will continue to complete all clinical, safety and immunological evaluations according to a defined schedule during the Treatment Period (see Appendix A).

### End of Treatment:

The Treatment Period is defined as time from the beginning of first study treatment to treatment exit. Treatment exit is defined as Week 24 or early termination. Subjects will enter the Post-Treatment Follow Up Period as described below, unless study treatment is discontinued prior to the completion of the treatment period. If the subject exits the treatment period before receiving all scheduled doses of study treatment because of:
Disease Progression: The subject will enter the post-treatment follow-up period, be followed for safety and then be followed for survival (see below).
Reasons other than Disease Progression: The subject will enter the post-treatment follow-up period and continue to be followed for safety and disease status assessment per Appendix A according to RECIST v1.1 (see below under “post-treatment follow-up period” for more information).
Investigator's decision, withdrawal of consent, lost to follow-up or death: The subject will have no further information collected, but the sponsor will retain and continue to use any data collected before the withdrawal of consent or date of last contact.

### Post-Treatment Follow-Up Period

Safety Follow-up: All subjects will be followed at least 30 days after End of Treatment for safety.
Long Term Follow-up: All subjects will be followed for survival approximately every three months [±2 weeks] for one year, until death, withdrawal of consent, loss to follow-up, or sponsor decision to close study, whichever comes first.
Information collected during LTFU will include all subsequent anti-cancer therapies (including the start/end date and reasons for changing regimens, best response on each regimen). The date/cause of death will also be collected.

### Duration of Treatment

Subjects will be treated for approximately 24 weeks during the Treatment Period, enter Post-Treatment Follow Up with a 30-day safety follow-up, and then followed for survival until death, withdrawal of consent, lost to follow-up, or sponsor decision to close study for up to 1 years. It is anticipated that recruitment for the initial 10 subjects will be completed within approximately 6 months and the entire study, including 1 year follow up period, will last approximately 2 years. Depending on whether additional subjects are enrolled in the event that there are safety or efficacy issues that need a larger pool of data, the study could last proportionally longer.

### End of Study

The End of Study (EOS) for a given subject is defined as when the subject has completed or discontinues from follow-up assessments as detailed in the respective Time and Events Schedule.

The end of study overall is defined as one year after the date of the last follow-up visit of the last subject who entered the follow-up period, or sponsor's decision to end the study earlier.

### Study Population

Prospective approvals of protocol deviations to recruitment and enrollment criteria, also known as protocol waivers or exemptions, are not permitted.

### Inclusion Criteria

Subjects are eligible to be included in the study only if all of the following criteria apply:
1. Adult females (18 years old and older)
2. Subjects with CA125-associated recurrent epithelial adenocarcinoma of ovarian, fallopian tube or peritoneal origin.
3. Have one of the eligible histologic epithelial cell types: serous adenocarcinoma, endometrioid adenocarcinoma, undifferentiated carcinoma, clear cell adenocarcinoma, mixed epithelial carcinoma, transitional cell carcinoma, malignant Brenner's Tumor, carcinosarcoma, or adenocarcinoma not otherwise specified (N.O.S.).
4. Previously treated FIGO Stage III and IV disease with up to 3 prior lines of therapy, including at least one platinum-based therapy. Each line of therapy should have been changed due to recurrence, progression, or toxicity. Maintenance therapy with bevacizumab, hormonal therapies and/or a PARP inhibitor is not considered a line of therapy.
5. Must have received prior platinum-based chemotherapy for first line ovarian cancer, however they must have been platinum sensitive for ≥6 after the most recent platinum-containing regimen prior to the start of study treatment.
6. Must have medical assessment consistent with prognosis for an expected survival of at least 6 months and be clinically appropriate to receive a 12-week hiatus from any cytotoxic treatment according to the best clinical judgement of the treating investigator.
7. Must have had an elevated serum CA125 >50 units/mL measured at screening within 28 days of start of study treatment.
8. Must have measurable disease, including identification of marker lesions, by radiographic or physical criteria suitable for evaluation according to RECIST v1.1 for documentation of disease response or progression.
9. Must have an ECOG Performance Status of 0 or 1.
10. Must have adequate organ function defined as:
   a. Absolute neutrophil count ≥1,500/μL
   b. Platelets ≥100,000/μL
   c. Hemoglobin 9 g/dL
   d. Total bilirubin ≤1.5×ULN (52.0 in subjects with known Gilberts syndrome) OR direct bilirubin ≤1×ULN
   e. LDH, SGOT and SGPT<2.5 times upper limits of normal
   f. Albumin >3.5 g/dL
   g. Serum creatinine s 1.5×upper limit of normal (ULN) or calculated creatinine clearance 30 mL/min using the Cockcroft-Gault equation
11. For women of childbearing potential, negative pregnancy test and willingness to avoid pregnancy by using a highly effective method of contraception from the first dose of study treatment to 60 days after last dose of study treatment.
12. Able to take oral medications.
13. Sign informed consent and authorization permitting release of personal health information.

### Exclusion Criteria

Subjects are excluded from the study if any of the following criteria apply:
1. Subject must not be simultaneously treated in any interventional clinical trial.
2. Subject must not have had major surgery ≤3 weeks prior to initiating protocol therapy and subject must have recovered from any surgical effects.
3. Subject must not have received investigational therapy ≤4 weeks, or within a time interval less than at least 5 half-lives of the investigational agent, whichever is shorter, prior to initiating protocol therapy.
4. Subject has had radiation therapy encompassing >20% of the bone marrow within 2 weeks; or any radiation therapy within 1 week prior to Day 1 of protocol therapy.
5. Subject must not have received a transfusion (platelets or red blood cells) s 2 weeks prior to first dose of study treatment.
6. Subject must not have received colony-stimulating factors (e.g., granulocyte colony-stimulating factor, granulocyte macrophage colony-stimulating factor, or recombinant erythropoietin) within 4 weeks prior initiating protocol therapy.
7. Subject has had any known Grade 3 or 4 anemia, neutropenia or thrombocytopenia due to prior chemotherapy that persisted >4 weeks and was related to the most recent treatment.
8. Subject must not have a serious, uncontrolled medical disorder, nonmalignant systemic disease, or active, uncontrolled infection or active infection causing fever. Examples include, but are not limited to, uncontrolled major seizure disorder, unstable spinal cord compression, superior vena cava syndrome, or any psychiatric disorder that prohibits obtaining informed consent. Subjects with chronic diseases that are well controlled (e.g., diabetes mellitus, hypertension [<150 sBP and <90 dBP]) are eligible.
9. Evidence of clinically significant cardiovascular and respiratory conditions including myocardial infarction within 1 year, uncontrolled or unstable angina, congestive heart failure (New York Heart Association Class Ill or IV), arrhythmia (Grade 2 or higher), chronic obstructive pulmonary disease, persistent asthma, or a history of asthma within 5 years.
10. Subject must not have any known history of myelodysplastic syndrome (MDS) or acute myeloid leukemia (AML)
11. Diagnosed or treated for another malignancy within 5 years before the first dose, or previously diagnosed with another malignancy and have any evidence of residual disease. Subjects with non-melanoma skin cancer or cervix carcinoma in situ are not excluded if they have undergone complete resection.
12. Subject must not have known, symptomatic brain or leptomeningeal metastases.
13. Have an active autoimmune disease (e.g., rheumatoid arthritis, SLE, ulcerative colitis, Crohn's Disease, MS, ankylosing spondylitis, thyroiditis) requiring continuing immune suppressive therapy.
14. Recognized immunodeficiency condition including cellular immunodeficiencies, hypogammaglobulinemia or dysgammaglobulinemia; subjects who have hereditary, or congenital immunodeficiency's (HIV infection, see below).
15. Chronically treated with systemic doses of immunosuppressive drugs such as cyclosporine, methotrexate, adrenocorticotropic hormone (ACTH) or immune suppressive monoclonal antibodies.
16. Chronic therapeutic corticosteroid use, defined as >5 days of prednisone or equivalent, with the exception of inhalers or those on a pre-planned steroid taper. (Note: Premedication with corticosteroids per institutional standard of care is allowed).
17. Any previous treatments with oregovomab.
18. Known allergy to murine proteins or hypersensitivity to any of the excipients of oregovomab or niraparib.
19. Have contraindications to the use of presser agents (e.g., SC epinephrine), notably monoamine oxidase inhibitor (MAOI) use.
20. Any of the following conditions (on-study testing is not required):
   a. Known HIV-infected subjects unless on effective anti-retroviral therapy with an undetectable viral load within 6 months, or
   b. Known or suspected hepatitis B if active infection (subjects with chronic hepatitis B infection must have an undetectable HBV viral load on suppressive therapy, if indicated; positive surface antibody alone is not an exclusion), or
   c. Known or suspected hepatitis C infection which has not been treated and cured unless currently on treatment with an undetectable viral load).
21. Unable understand, and/or unwilling to sign a written consent form which must be obtained prior to treatment.

### Screen Failures

Subjects who sign an informed consent but fail to be started on treatment for any reason will be considered screen failures. The reason for not being started on treatment will be collected. Minimal information collected on screen failures will include demography, reason for screen failure and any serious adverse event (SAE) that occurred during the Screening Period.

### Subject Replacement/Re-Screening

Subjects who receive at least one dose of either study drug will not be replaced. A subject who is screened but does not receive either study drug may be replaced.

Subjects who fail screening on any single criterion where there is the prospect of their subsequently becoming eligible, may be re-screened once, using the same screening number.

### Treatment of Subjects

Study Intervention is defined as any investigational intervention(s), marketed product(s), or medical device(s) intended to be administered to study subjects according to the study protocol.
“Study drugs” are the experimental biologic oregovomab and PARP inhibitor, niraparib
“Study Treatment” is referring to study drugs taken alone or in combination and administered at the frequencies outlined in the Time and Events Schedule (Appendix A).

The details about the study drugs are shown in Table 3.

### Study Drug Information

TABLE 3
Study Drugs

| | Investigational | |
|---|---|---|
| Intervention | Oregovomab | |
| Name | (MAb-B43.13) | PARP inhibitor Niraparib |
| Type | Biologic | Drug |
| Dose | Powder for injection | Tablets |
| Formulation | | |
| Dosage | 2 mg | 300 mg (3 × 100 mg tablets) |
| Level(s) | | |
| Route of | IV Infusion | Oral |
| Admin. | | |
| Sourcing | Provided centrally by | Provided centrally by Sponsor. |
| | Sponsor. | All dispensing and |
| | All dispensing and | accountability records should |
| | accountability | be stored in accordance with |
| | records should be stored | the institution regulations. |
| | in accordance with the | The pharmacist will dispen |
| | institution regulations. | se study drug for each subject |
| | The pharmacist will | according to the protocol and |
| | dispense study drug for | storage and handling manual, |
| | each subject according to | if applicable. |
| | the protocol and storage | |
| | and handling manual, if | |
| | applicable. | |
| Packaging | Oregovomab will be | Niraparib is supplied by GSK |
| and | provided in a box | in high-density polyethylene |
| Labeling | containing vials. Each | (HDPE) bottles with 30 tablets |
| | vial and box will be | of 100 mg each and with |
| | labeled as required per | child-resistant plastic closure |
| | country requirement. | tops. The study drug will be |
| | | open-label and will not be |
| | | subject-specific. Detailed |
| | | information on the product |
| | | can be found in the Niraparib |
| | | Storage and Handling |
| | | Guidelines. |
| Storage | Store un-reconstituted | The study drug must be stored |
| | vials at 2° C. to 8° C. | at room temperature, in a |
| | Following reconstitution, | securely locked area, |
| | oregovomab MAb-B43.13 | accessible to authorized |
| | solution may be stored at | personnel only. |
| | room temperature (20-25° | |
| | C.) for up to 4 hours. | |
| Current/ | OvaRex | Zejula |
| Former | | |
| Names or | | |
| Alias | | |

### Allocation to Treatment

No randomization or stratification will be performed in this study.

### Study Drug Administration

All subjects will receive the combination of niraparib and oregovomab. See the Pharmacy Manual for more information.

On clinic days, subjects will take niraparib first, then 1-2 hours later, oregovomab will be administered. Subjects will be observed for 1-2 hours after the end of the oregovomab infusion during which time they will be monitored for the possible appearance of the symptoms of allergy, including anaphylaxis, which may present as: fever, chills, dizziness, nausea, headache, diarrhea, dyspnea, tachypnea, hypotension, and tachycardia. With multiple doses of oregovomab or prior exposure to murine proteins, there is a possibility of severe or life-threatening allergic reaction.

If such a reaction to occur, a physician must be immediately available for treatment of the subject; epinephrine, antihistamines and corticosteroids must be available for use.

If niraparib is delayed, oregovomab must be similarly delayed and given in the same sequence defined above.

### Niraparib

Niraparib will be administered as a flat-fixed, continuous daily dose according to Table 3 below. Niraparib tablets should be swallowed whole and not opened, crushed or chewed. Food does not significantly affect the absorption of niraparib; therefore, niraparib may be taken without regard to meals. Subjects should take doses at approximately the same time each day. Bedtime administration may be a potential method for managing nausea.

Vomited doses should not be made up.

If a subject misses a dose (greater than 12 hours from normal dosing time) of niraparib, they should skip that dose and take their next dose at its regularly scheduled time.

If niraparib is dose reduced, subjects should be instructed to continue using their current supply at their new dose until their supply has been exhausted.

Subjects must be instructed to return unused study drugs to the site at discontinuation or completion of treatment. The site personnel must ensure that the appropriate dose of each study drug is administered and that the drug accountability is performed and documented.

TABLE 3
Niraparib Dosing

| Patient Weight and Platelet | |
|---|---|
| Count at Baseline | Starting Dose |
| ≥77 kg and ≥150,000 μL | 300 mg (3 × 100 mg tablets) |
| | 3 tablets daily |
| <77 kg and/or <150,000 μL | 200 mg (2 × 100 mg tablets) |
| | 2 tablets daily* |

*For subjects whose starting dose is 2 tablets once daily, escalation to 3 tablets once daily is permitted if no treatment interruption or discontinuation was required during the first 2 cycles of therapy.

### Weeks 1 Through 12:

The daily dose of niraparib will be (3×100 mg tablets) taken orally from Week 1 Day 1 to the end of Week 12. Subjects whose baseline weight is <77 kg or baseline platelet count is <150,000 μL, the daily dose of niraparib will be 200 mg (2×100 mg tablets of niraparib).
Subjects must bring their pill bottles and completed pill diary to every clinic visit for clinic staff to perform drug accountability.

### Weeks 13 Through 24:

Subjects who have stable disease [SD], partial response [PR], or complete response [CR], as per RECIST v1.1, at Week 12, may continue to take niraparib for an additional 12 weeks (total 24 weeks) or switch to alternative anti-cancer regimen of Investigator's choice.

### Oregovomab

A dose of 2 mg will be administered via IV infusion in 50 mL of normal saline over 20 minutes±5 minutes during clinic visits at Day 1 of Week 1, Week 4, Week 7, Week 12, and Week 20.

All subjects, regardless of therapy administered post Week 12, are required to take the 5th dose of oregovomab at Week 20 visit.

### Dose Modification

### Niraparib

Dose interruption and/or modification of niraparib may be implemented due to nonhematologic or hematologic toxicities per the Investigator's judgement after Cycle 1.

Treatment must be interrupted for any nonhematologic Common Terminology Criteria for Adverse Events (CTCAE) Grade 3 or 4 AE that the Investigator considers to be related to administration of niraparib (Table 5). If the nonhematologic toxicity is appropriately resolved to baseline or Grade 51 within 4 weeks (28 days) of the dose interruption period, the subject may restart treatment with niraparib but with a dose level reduction, if prophylaxis is not considered feasible (see Table 4). If the event recurs at similar or worse grade, treatment should be interrupted again and, upon resolution, a further dose reduction must be made according to Table 4.

If the toxicity requiring dose interruption has not resolved completely or to CTCAE Grade 1 during the maximum 4-week (28-day) dose interruption period, and/or the subject has already undergone a dose reduction to a minimum dose of 100 mg QD, the subject must permanently discontinue treatment with niraparib.

The dose interruption and modification criteria for niraparib for hematologic parameters will be based on blood counts and are outlined in Table 6. If the hematologic toxicity has not recovered to the specified levels within 4 weeks (28 days) of the dose interruption period, the subject must permanently discontinue treatment with niraparib.

For subjects whose initial dose is 3 tablets daily (300 mg/day), dose reductions to 2 tablets daily (200 mg/day) and subsequently to 1 tablet daily (100 mg/day) will be allowed. No further dose reduction will be allowed.

For subjects whose initial dose is 2 tablets (200 mg/day), dose reduction to 1 tablet once daily (100 mg/day) will be allowed. No further dose reduction will be allowed.

TABLE 4
Recommended Dose Modifications for Adverse Reactions

| | Initial Dose: | Initial Dose: |
|---|---|---|
| Dose level | 3 tablets per day | 2 tablets per day |
| Starting | 3 tablets QD (300 mg/day) | 2 tablets QD (200 mg/day) |
| dose | | |
| First dose | 2 tablets QD (200 mg/day) | 1 tablet QD (100 mg/day) |
| reduction | | |
| Second dose | 1 tablet QD (100 mg/day) | NA |
| reduction | | |

TABLE 5
Niraparib Dose Modifications for Non-hematologic Adverse Reactions

| Abnormality | Intervention |
|---|---|
| Non-hematologic CTCAE ≥ | Withhold niraparib for a maximum |
| Grade 3 adverse reaction where | of 28 days or until resolution of |
| prophylaxis is not considered | adverse reaction. |
| feasible or adverse reaction | Resume niraparib at a reduced dose. |
| persists despite treatment | |
| CTCAE ≥ Grade 3 treatment- | Discontinue niraparib. |
| related adverse reaction lasting | |
| more than 28 days while subject | |
| is administered niraparib 100 | |
| mg/day | |

TABLE 6
Niraparib Dose Modifications for Hematologic Toxicity

| | |
|---|---|
| Laboratory | |
| Abnormality | Intervention |

Monitor complete blood counts weekly for the first month, monthly
for the next 11 months of treatment, and periodically after this time.

| | |
|---|---|
| Platelet count | First occurrence: |
| <100,000/μL | Withhold niraparib for a maximum of 28 days |
| | and monitor blood counts weekly until platelet |
| | counts return to ≥100,000/μL. |
| | Resume niraparib at same or reduced dose per |
| | Table 3. |
| | If platelet count is <75,000/μL, resume niraparib |
| | dat a reduced ose per Table 3. |
| | Second occurrence: |
| | Withhold niraparib for a maximum of 28 days |
| | and monitor blood counts weekly until platelet |
| | counts return to ≥100,000/μL. |
| | Resume niraparib at a reduced dose per Table 3. |
| | Discontinue niraparib if the platelet count has not |
| | returned to acceptable levels within 28 days of |
| | the dose interruption period, or if the subject has |
| | already undergone dose reduction to 100 mg QD. |
| Neutrophil count | Withhold niraparib for a maximum of 28 days |
| <1,000/μL | and monitor blood counts until neutrophil counts |
| | return to ≥1,500/μL. |
| | Resume niraparib at a reduced dose per Table3. |
| | Discontinue niraparib if neutrophil level has not |
| | returned to acceptable levels within 28 days of |
| | the dose interruption period, or if the subject has |
| | already undergone maximum dose reductions per |
| | Table 3. |
| Hemoglobin | Withhold niraparib for a maximum of 28 days |
| ≤8 g/dL | and monitor blood counts until hemoglobin |
| | returns to ≥9 g/dL. |
| | Resume niraparib at a reduced dose per Table 3. |
| | Discontinue niraparib if hemoglobin has not |
| | returned to acceptable levels within 28 days of |
| | the dose interruption period, or if the subject has |
| | already undergone maximum dose reductions per |
| | Table 3. |
| Hematologic adverse | For subjects with platelet count ≤10,000/μL, |
| reaction requiring | platelet transfusion should be considered. If there |
| transfusion | are other risk factors such as co-administration of |
| | anticoagulation or antiplatelet drugs, consider |
| | interrupting these drugs and/or transfusion at a |
| | higher platelet count. |
| | Resume niraparib at a reduced dose per Table 3. |
| Confirmed diagnosis | Permanently discontinue niraparib. |
| of MDS or AML | |

In the case of thrombocytopenia, following the first occurrence, resumption of therapy may occur at the same dose or 1 dose level lower when the hematologic toxicity has resolved. Subsequent occurrences should trigger dose reduction upon resumption of therapy. If the platelet count has not reverted within 28 days of interruption to ≥100,000/μL, then study treatment should be discontinued.

If dose interruption and/or modification is required at any point during study treatment because of hematologic toxicity, weekly blood draws for complete blood count (CBC) will be monitored until the AE resolves to the specified blood count levels. To ensure the safety of the new dose, weekly blood draws for CBC will be required for an additional 4 weeks after the AE has resolved, after which monitoring every 4 weeks may resume. CBC monitoring will continue every 4 weeks (ie, monthly) for the next 11 months of treatment, and periodically after this time.

Any subject requiring transfusion of platelets or red blood cells (≥1 unit) must undergo a dose reduction upon recovery if study treatment is resumed.

If a diagnosis of MDS/AML is confirmed by a hematologist, the subject must permanently discontinue study treatment.

For surgical procedures while on study treatment, up to 4 weeks (28 days) of study treatment interruption is allowed.

### Oregovomab

For oregovomab, dose modifications or dose reductions are not permitted. If needed in the case of suspected infusion reactions and at the investigator's discretion, oregovomab infusion may be interrupted for up to 2 hours prior to resuming the infusion. The infusion time may also be increased to 40-60 minutes, and standard antihistamine, corticosteroid and/or epinephrine therapy may be used as necessary as per institutional guidelines for infusion reactions.
Infusion reactions causing interruption of oregovomab must recover completely or to CTCAE Grade 1 within 2 hours.
Subjects can be cautiously re-challenged with or without premedication with corticosteroid and/or antihistamine in consultation with the Medical Monitor.

In the event where a CTCAE Grade 3 AE to occur that cannot be directly attributed to either treatment, the subject must recover completely or to CTCAE Grade 1 within 7 days, except for the following:
Alopecia of any CTCAE grade
Any other skin toxicity recovered to CTCAE Grade ≤2 with adequate supportive care measures within 2 weeks after last dose.
Fatigue CTCAE Grade 3 recovered to CTCAE Grade ≤2 within 2 weeks after last dose.
Grade 3 AEs of headache, insomnia, diarrhea, nausea and vomiting that resolve to ≤Grade 2 within <72 hours.
Any other non-hematologic or non-cardiac CTCAE Grade 3 or 4 toxicity that resolved to Grade ≤1 or pre-existing grade at study entry by the next dose.

If not able to tolerate oregovomab treatment after interrupting treatment with adequate supportive care and reducing the rate of infusion, the subject will not receive any further treatment with oregovomab. However, the subject may continue being treated with protocol-directed niraparib and be followed for survival.

### Blinding

This is not a blinded study.

### Preparation/Handling/Storage/Accountability

The investigator or designee must confirm appropriate temperature conditions have been maintained for all study drugs received and any discrepancies are reported and resolved before use of the study drug.

Only subjects enrolled in the study may receive study treatment and only authorized site staff may supply or administer study treatment. Each study drug must be stored in a secure, environmentally controlled (e.g., refrigerated), and monitored (manual or automated) area in accordance with the labeled storage conditions with access limited to the investigator and authorized site staff.

The investigator (or designee at the institution) is responsible for study drug accountability, reconciliation, and record maintenance (e.g., receipt, reconciliation, and final disposition records).

For preparation, dose administration instructions for study drugs, and for instructions on study drug destruction please see the Pharmacy Manual.

### Study Drug Compliance

Subject compliance with study drugs will be assessed at each visit. Deviation(s) from the prescribed dosage regimen will be recorded in the source and the CRF by the investigator and/or study personnel at each site.

### Prior and Concomitant Therapies

### Permitted Concomitant Therapies

Any medication or inactivated vaccine (including over the counter or prescription medicines, vitamins, and/or herbal supplements) deemed for supportive care and safety of the subject received at the time of enrollment, or receives during the study, must be recorded in the CRF along with reason for use, dates of administration including start and end dates, and dosage information including dose and frequency. This may include, but is not limited to, the following:
Medications to prevent or treat nausea or vomiting.
Symptomatic treatments of tumor-associated symptoms, bone modifiers and luteinizing hormone-releasing hormone (LHRH) agents
Anti-diarrheal medications for subjects who develop diarrhea.
Pain medication to allow the subject to be as comfortable as possible.
Immunosuppressive agents to treat suspected immune response related AEs.
Medications to treat chronic diseases (e.g., hypertension, hypercholesterolemia, etc.)
Localized radiotherapy and treatment with bisphosphonates for pre-existing, painful bone/liver metastases are permitted. The radiotherapy must be listed on the CRF.
Oxygen therapy and blood products or transfusions.
Limited-field palliative radiotherapy may be allowed as concomitant therapy. Local therapies administered during the study drug must be listed on the CRF.
Inactivated vaccines.
Nutritional support or appetite stimulants
   For oregovomab: The use of pre-infusion medication and post-infusion medication is allowed. Specifically:
Subjects who have experienced infusion reaction(s) to oregovomab during prior infusion(s), in subsequent drug administrations may be pretreated with corticosteroids (such as dexamethasone), antihistamines (such as diphenhydramine), and H2 antagonists (such as cimetidine or ranitidine) at least 30 minutes prior to oregovomab.
If allergic reactions are observed during infusion and up to 2 hours post-infusion, standard epinephrine, corticosteroid, and antihistamine therapy according to institutional guidelines may be used.

For niraparib: For more information, please refer to the Niraparib IB. The subject must notify the Investigator about any new medications taken after the start of the study drug. All medications (other than study drug) and significant non-drug therapies (including physical therapy, herbal/natural medications and blood transfusions) administered during the study must be listed in the CRF.

Other concomitant medication not listed above may be considered on a case-by-case basis by the investigator in consultation with the Medical Monitor if needed.

### Prohibited Concomitant Therapies or Therapies Requiring Caution

Subjects may not use any investigational drugs or any of the therapies listed in the Exclusion Criteria concomitantly with the investigational agents. Because certain drugs may interfere with the immunological mechanisms mediating the effect of the study drugs, it is recommended that the following medications not be given to subjects during the treatment period:

### Prohibited Therapies for Oregovomab:

ACTH
Cyclosporine
Azathioprine
Live vaccines such as Yellow Fever vaccine—28 days prior to administration of first dose of study drug up until at least 90 days after the last dose of study drug
Strong CYP3A4 inhibitor (e.g., Clarithromycin, telithromycin, nefazodone, itraconazole, ketoconazole, atazanavir, darunavir, indinavir, lopinavir, nelfinavir, ritonavir, saquinavir, tipranavir)
Strong inducers of CYP3A4 or of CYP2C8 (e.g., rifampicin, phenytoin, carbamazepine)
Biological and hormone therapy
Nephrotoxic drugs
Systemic doses of immunosuppressive drugs (such as corticosteroids, methotrexate, or immune suppressive monoclonal antibodies) where the subject is treated chronically.
Other investigational drugs within 30 days of Week 1 Day 1 or during the study

Because certain drugs may interfere with the immunological mechanisms mediating the effect of oregovomab, it is recommended that the following medications not be given to subjects during the treatment period:
Additional acute use of corticosteroids (such as dexamethasone), other than corticosteroids used for chemotherapy premedication and hypersensitivity reactions, should be avoided if possible, particularly at the time of oregovomab administration, but may be used at the discretion of the treating physician.
Chronic systemic corticosteroids, >5 days of prednisone or equivalent (e.g., dexamethasone, prednisone, etc.). However, corticosteroids administered by inhalation for the treatment of asthma or administered topically are allowed.
Radiation therapy
Vitamin A
Antifungal agents (e.g., ketoconazole, Miconazole)
Doxorubicin
Myelosuppressive agents
Aminoglycosides
Monoamine oxidase inhibitors (MAOI) should be avoided, if possible, as they may interact with presser agents (e.g., SC epinephrine), which may cause severe hypertension.
Subjects who have been treated with chronic immunosuppressive drugs in the past, must have been off treatment for at least 8 weeks before starting on study.

### Prohibited Therapies or Therapies Requiring Caution for Niraparib:

The following medications are prohibited while receiving protocol therapy:
Systemic anticancer or biological therapy.
Immunotherapy.
Chemotherapy.
Investigational agents other than niraparib and oregovomab
Radiation therapy encompassing >20% of the bone marrow is prohibited within 2 weeks prior to Day 1 and during study treatment. Note: Palliative radiation therapy to a small field >1 week prior to Day 1 of study treatment may be allowed.
Any surgery that involves tumor lesions. Note: Administration of radiation therapy or surgery done that involves tumor lesions will be considered as disease progression at the time the procedure is performed.
Niraparib weakly induces Cytochrome P450 (CYP)1A2 in vitro and is a relatively poor substrate for P-glycoprotein (P-gp); therefore, investigators are advised to use caution with the substrates for CYP1A2 with a narrow therapeutic range, i.e. theophylline and tizanidine.
Prophylactic cytokines (i.e., granulocyte colony-stimulating factor [GCSF]) should not be administered in the first cycle of the study but may be administered in subsequent cycles according to current American Society of Clinical Oncology (ASCO) guidelines.

### Rescue Medicine

Rescue medication, as part of study treatment, will not be provided by the sponsor (or its designee) in this study.

Changes to Anti-Neoplastic Therapy Post Week 12 and Post Week 24 or with Uncontrolled Disease Progression

Clinical judgment of the treating physician should guide the management plan of each subject based on individual benefit/risk assessment. The responsible Investigator should institute appropriate diagnostic and therapeutic measures and should keep the subject under observation for as long as is medically indicated, as per standard of care. The 12-week period to first radiographic evaluation is a period of immune priming that may or may not include disease control activity from the experimental combination. In the absence of disease control, niraparib should be discontinued and the deferred next line anti-neoplastic treatment of choice initiated for study subjects. A final immunization at week 20 should be provided to all patients.

The anti-neoplastic therapy(ies) can be initiated post week 12 evaluation and administered according to investigators best practice, and all information during the week 12-24 interval following niraparib discontinuation will be collected along with the start/end date. Therapies initiated immediately post week 24 will also be collected for start date and followed in the follow up survey. This may include medications, radiotherapies or surgeries provided to subjects since discontinuing study drug. For patients experiencing uncontrolled disease progression at any time during the protocol clinical judgment and premature discontinuation from the protocol if indicated should be implemented according to the clinical judgment of the treating physician.

### Study Assessments and Procedures

Study procedures and their timing are summarized in the Time and Events Schedule with an “x” indicating when the assessments are performed.

Protocol waivers or exemptions are not allowed. Immediate safety concerns should be discussed with the sponsor immediately upon occurrence or awareness to determine if the subject should continue or discontinue study drug.

It is expected that all data obtained from these assessments listed in the Time and Events Schedule must be supported in the subject's source documentation.

Allowed visit windows are specified as follows:
There will be no visit window for Week 1 Day 1.
A general ±3-day window is permitted on assessments to take into account scheduling over public holidays, if not explicitly specified otherwise.
Imaging Evaluations must be performed as outlined in the Time and Events Schedule.

### Pre-Treatment Procedures and Assessments

Subjects with platinum sensitive recurrent ovarian cancer will be screened for eligibility during the period 28 days prior to Week 1 Day 1. During this time, the inclusion and exclusion criteria will be assessed as described in herein and all screening assessments, laboratory tests, and procedures will be performed.

Results of all screening/baseline evaluations must be reviewed by the investigator or his/her designee prior to enrolling subject into the study in order to assure that all inclusion and exclusion criteria have been satisfied. All study subjects must be thoroughly informed about all aspects of the study, including the study agents, visit schedule, required evaluations, and all regulatory requirements for informed consent.

For laboratory evaluations used to determine eligibility, a repeated evaluation within the screening window is permitted for screening results out of the defined range before screen failing the subject. If the repeated laboratory result meets the criteria, that result may be used to determine eligibility. If the repeated laboratory result does not meet the criteria, the subject will be considered a screening failure.

Any imaging assessments already completed during the regularwork-up of the subject within 28 days prior to starting study treatment can be considered as the baseline images for this study.

A list of procedures to be performed at the time of screening is summarized in the Time and Events Schedule.

### Informed Consent and Confirmation of Participation

Potentially eligible subjects will be evaluated for participation and asked to sign the Informed Consent Form (ICF) prior to executing screening procedures to confirm they satisfy all inclusion and exclusion conditions.

When a subject is considered eligible for enrolment into the study, a Patient Registration Form should be completed and sent to OncoQuest Pharmaceuticals Inc. or its designee. The OncoQuest Pharmaceuticals Inc. or its designee will assign the subject number. Once assigned, the subject number must not be reused for any other subject and the subject number for that individual must not be changed, even if the subject is re-screened. If the subject fails to be enrolled or start treatment for any reason, the reason will be entered into the Screen Failure CRF.

### Subject Demography

Data that will be collected at screening includes, but is not limited to, the following:
Sex/gender at birth
Date of birth (or age or year of birth only, if required based on location regulations)
Race and ethnicity (if available)
Subject Initials (if collection is allowed by local regulations)

### Medical History

Complete medical history will be collected during screening and will include important medical, surgical, and allergic conditions from the subject's medical history which could have an impact on the subject's evaluation/current medical conditions (e.g., all relevant current medical conditions which are present at the time of signing informed consent).

The medical history must include details on the initial diagnosis and extent of ovarian cancer, including, but not limited to, date of diagnosis of disease, staging at initial diagnosis, histology/cytology, histological grade, any metastatic sites, primary site of cancer, etc.

If medications are taken for ongoing non-cancer medical conditions, they must be recorded on the concomitant medications CRF.

All prior surgeries, their reason and description, date of procedure and result will be captured.

### Relevant Cancer History

Relevant cancer history must be recorded for each subject in the CRF, including, but not limited to the following:
Primary site of cancer
Staging System
Staging at initial diagnosis and at study entry
Date of first recurrence/progression
Most recent relapse/progression
Histological grade
Predominant histology/cytology
Presence of metastatic sites (and if sites available, locations)

### FIGO Staging

The FIGO staging system is a classification system for ovarian cancers established by the International Federation of Gynecology and Obstetrics (Berek et al., 2018). See Appendix B for more information. FIGO Staging will be recorded in the subject's CRF.

FIGO stage will be documented during screening and prior to the first dose of study treatment.

Note: Eligible subjects must have FIGO Stage III or IV disease to be included in the study.

### Prior Anti-Cancer Therapies

All prior anti-cancer therapies must be recorded on the appropriate CRF including surgical interventions (including debulking surgeries, whether optimal or suboptimal and primary or interval) and chemo-, biologic-, immunologic- and radiation-therapies provided as treatment for cancer prior to the administration of study drug, including prior PARP inhibitors.

There are no restrictions against previous PARP inhibitor therapies usage.

### BRCA1, BRCA2, and HRD Status

During Screening, historical sample collection of BRCA1, BRCA2 mutations and HRD Status must be collected from subject's medical history and recorded on the appropriate CRF page.

### Other Baseline Assessments

Additional screening/baseline assessments will be performed according to the respective Time and Events Schedules (Appendix A). This includes, but is not limited to, the following:
1. β-HCG test
2. Prior and concomitant medications
3. CA-125

### Safety Assessments

Significant findings that were present prior to the signing of informed consent must be included in the Medical History page on the subject's CRF. Significant new findings that begin or worsen after informed consent must be recorded on the Adverse Event page of the subject's CRF.

### Vital Signs

Vital signs (heart rate [beats per minute], blood pressure [systolic and diastolic blood pressure] taken while seated after at least 3 minutes of resting, respiratory rate [per minute] and temperature [in Celsius]) will be recorded within 30 minutes of start of oregovomab administration and at the completion of oregovomab within 1 hour of stopping administration. On days where oregovomab administration is not done during a clinic visit, vital signs should be taken according to institutional standards.

Height in centimeters (cm) will be measured at screening only.

Body weight (in kilograms) in indoor clothing, but without shoes, will be measured at screening and at subsequent time points as specified in the Time and Events Schedule.

### Physical Examination

Physical examination will be performed to assess effects of the study drug administration. At selected visits, physical exams will be performed according to the schedule given in the Time and Events schedule.

An assessment should be performed of the head and neck, eye, ear, nose & throat (EENT), cardiac, respiratory, abdomen, extremities, pelvic, skin and lymph nodes to identify normal or abnormal findings. A description and severity of any abnormalities must be noted.

### ECOG

The performance status will be assessed according to the ECOG performance status scale (Oken et al., 1982) following the schedule given in the Time and Events Schedule.

TABLE 7
ECOG Performance Status

| Grade | ECOG status |
|---|---|
| 0 | Fully active, able to carry on all pre-disease performance without |
| | restriction |
| 1 | Restricted in physically strenuous activity but ambulatory and |
| | able to carry out work of a light or sedentary nature e.g., light |
| | housework, office work |
| 2 | Ambulatory and capable of all self-care but unable to carry out |
| | any work activities. Up and about more than 50% of waking |
| | hours Capable of only limited self-care, confined to bed or chair |
| | more than 50% of waking hours |
| 4 | Completely disabled. Cannot carry on any self-care. Totally |
| | confined to bed or chair |
| 5 | Dead |

### Laboratory Examinations

All laboratory assessments described in Table 8 will be done locally at the site and will be taken according to the Time and Events schedule. Local laboratories will be used to qualify subjects for entry into the study or used in cases of a safety concern during the study.

Any post-baseline laboratory value that is found to be clinically significant will be evaluated by the Investigator for causal relationship to the administration of the study drugs and any medically appropriate action will be taken. Subjects experiencing adverse events will be monitored until all symptoms are resolved. Abnormal laboratory values will also be graded according to CTCAE v5.0. Abnormal values will be recorded in the CRF. Any laboratory parameters that are outside the normal range and are significantly different from the baseline values will be repeated as necessary until the abnormality has been resolved or the etiology clarified to the Investigators satisfaction.

Any unscheduled local laboratory assessment(s) may be performed if medically indicated to document a (potential) adverse event or for decision making (e.g., disease progression, etc.). Local laboratory samples will be collected and analyzed as per institutional requirements. Values and normal range(s) must be entered into the local laboratory CRF.

TABLE 8
List of Clinical Laboratory Parameters

| Category | Test Name |
|---|---|
| Hematology: | RBC, hemoglobin, hematocrit, WBC and differential |
| | (including neutrophil, lymphocytes, monocyte, |
| | eosinophil, basophil), and platelets |
| Biochemistry: | Glucose, sodium, potassium, chloride, magnesium, |
| | calcium, CO2/bicarbonate, creatinine, SGOT/AST, |
| | SGPT/ALT, GGT, alkaline phosphatase (ALP), |
| | phosphorus, LDH, bilirubin (total and direct), urea (or |
| | BUN), total protein, albumin. In case of pathological |
| | CPK further evaluation (eg, at least by Troponin assays, |
| | CK-MM, CK-MB, ECG exam) must be performed and |
| | the findings documented. |
| | GFR will be estimated by the Cockcroft-Gault formula |
| | utilizing serum creatinine values. |
| Coagulation: | PT, INR, and APTT |
| Urinalysis | pH, glucose, erythrocytes, leukocytes, protein, color, |
| and Urine | ketones, and nitrite will be analyzed by dipstick. If |
| Dipstick: | dipstick is abnormal then perform microscopic Panel |
| | (Red Blood Cells, White Blood Cells, Casts, Crystals, |
| | Bacteria, Epithelial cells); If applicable, urine pregnancy |
| | testing. |
| Thyroid: | Screening only: TSH (Thyroid Stimulation Hormone), |
| | Free T3 and Free T. At the subsequent visits as indicated: |
| | TSH only; If TSH is abnormal, test Free T3 and Free T4 |
| Serum and | If applicable, hCG test at time points indicated in the |
| Urine | Time and Events Schedule. |
| Pregnancy | |
| Tests: | |

### Pregnancy and Assessments of Fertility

Pregnancy testing is not required for subjects who are determined to be post-menopausal. Women are considered post-menopausal and not of childbearing potential if they have had 12 months of natural (spontaneous) amenorrhea with an appropriate clinical profile (i.e., age appropriate history of vasomotor symptoms) or have had surgical bilateral oophorectomy (with or without hysterectomy), total hysterectomy or tubal ligation at least six weeks prior to starting study treatment. In the case of oophorectomy alone, only when the reproductive status of the woman has been confirmed by follow up hormone level assessment is she considered not of child bearing potential.

For women of childbearing potential, they must complete a serum pregnancy test (R3-HOG) within 7 days prior to the first dose of study treatment and at the treatment exit. Urine pregnancy tests will be performed throughout the treatment period as indicated in the Time and Events Schedule. If a subject becomes pregnant during the treatment period, she must be discontinued from study treatment.

Male partners of participating women of childbearing potential must use a condom during intercourse while the female partner is taking the study treatment and for at least 30 days after the female partner stops study treatment.

### 12-Lead Electrocardiogram

A standard 12-lead electrocardiogram (ECG) will be performed after at least 5 minutes of resting supine at Screening, end of Week 12, and end Week 24 visits according with institutional guidelines. If a subject discontinued before reaching Week 24, ECG should be performed during the EOT visit. Interpretation of the tracing must be made by a qualified physician and documented on the subject's ECG eCRF page. Each ECG tracing should be labeled with the study number, subject initials (where regulations permit), subject number, date, and kept in the source documents at the study site.

### Imaging Studies

Imaging evaluation (CT/MRI), and the radiographic reports will be collected for disease assessment and will be done at Baseline, Week 12, and Week 24 visits using RECIST v1.1. Any subsequent imaging will be done according to institutional guidelines. Subjects will be treated until treatment exit at Week 24, discontinuation due to disease progression, lost to follow-up, withdrawal of consent, treatment with another anti-cancer drug, or death.

### Pharmacodynamic and Biomarker Assessments

No pharmacodynamic measurements will be conducted for this study. All biomarker assessments listed are exploratory in nature.

TABLE 9
Biomarker Assessments

| Humoral immune | Humoral response to oregovomab |
|---|---|
| Characterizations: | (Human Anti-Mouse Antibody (HAMA)) |
| Tumor Marker: | CA-125 |

### Early Humoral Immune Response

Early humoral immune response will be assessed by measuring HAMA at the time points specified in the Time and Events Schedule (Appendix A). Serum sample collection, processing and measurement of HAMA should be collected at site and sent to the reference laboratory according to the instructions in the laboratory manual. Early Humoral Response is defined as a value greater than 74 ng/ml that increased from baseline. The ratio will be calculated from the ratio of week 7 and baseline samples will also be calculated.

### CA-125

Blood samples to evaluate CA-125 will be collected according to the Time and Events Schedule (Appendix A).

The treatment of subjects with an anti-CA-125 murine antibody, B43.13 (oregovomab), may lead to falsely elevated or depressed CA-125 levels, depending on the assay utilized to perform testing during the Treatment Period. Therefore, no CA-125 measurements should be drawn within 15 days following an oregovomab infusion. Similarly, due to competition with a detector antibody in some commercially available CA-125 assays, the potential presence of HAMA could interfere with CA-125 measurement. Therefore, CA-125 determinations will be performed using an assay that is insensitive to the presence of HAMA (Abbott Architect i1000SR or equivalent).

### NMLR As A Prognostic Factor

The ratio of neutrophils plus monocytes to lymphocytes as calculated from the baseline pretreatment complete blood count with differential will associated with clinical outcomes.

### Samples for Future Evaluation of Changes in Cellular Immunity

Each patient will have 20 ml of whole blood collected at baseline, week 12 and week 24 and express shipped to the central immunology lab according to the instructions in the laboratory manual and using the provided kit. These samples will be separate to isolate PBMC and for cryopreserved at the central lab for future evaluation of antigen specific cellular immunity and immune phototyping.

### Pharmacokinetics

PK will not be collected in this study.

### Efficacy Assessments

### Tumor Assessments

Disease Control Rate (DCR) as determined by CR, PR and SD will be determined by the investigator according to RECIST v1.1 at weeks 12 and 24 (EA Eisenhauer et al., EJC 45 (2009) 228-247). The same method of assessment and the same technique should be used to characterize each individual and reported lesion at baseline and during follow up.

### Determination of Disease Progression

Disease progression will be assessed at the site and will be based on RECIST v1.1 at weeks 12 and 24. The response to second line therapy at week 24 will be compared to week 12 as well as to Baseline (EA Eisenhauer et al., EJC 45 (2009) 228-247). The decision regarding subject management will remain with the local Investigator.

Diagnosis of possible progression would be made on the basis of imaging, or other objective clinical findings. Objective clinical findings of progression could include palpable lesions, evidence of appearance of or worsening of pleural effusion or ascites requiring histological confirmation, pelvic and/or abdominal pain or other multi-system symptoms, preferably confirmed through imaging.

The date of achieving definitive disease progression will be used to calculate PFS. If an off-schedule imaging assessment is performed to confirm response or if progression is suspected, subsequent imaging assessments should be performed in accordance with the original imaging schedule.

### Exploratory Efficacy Endpoints and Analyses Measurement

The following exploratory tumor response measurements by selected intermediate clinical endpoints will be performed: PFS, TFST, and overall response rate (ORR) determined by CR and PR for second line chemotherapy at week 24 relative to week 12.CA-125 examination related to exploratory efficacy endpoint measurements will be performed.

### Missed Visits

In order to maintain integrity of study visits, every attempt will be made to adhere to the original visit schedule including all required testing and evaluations. For missed visits, these should be re-scheduled as soon as possible, unless the re-scheduled visit would be within two weeks of the next scheduled visit. In this case the missed visit will not be completed, the next visit will occur at the time originally scheduled. All study visits are to be scheduled based on the treatment schedule.

### Temporary or Permanent Discontinuation of Study Treatment

### Oregovomab

In rare instances, it may be necessary for a subject to permanently discontinue study treatment. If study drug is permanently discontinued, the subject will remain in the study to be evaluated for safety, post treatment efficacy follow-up in absence of disease progression, and survival, unless consent is withdrawn, or subject is lost to follow-up.

If a subject decides to discontinue from the study treatment, the investigator should make a reasonable effort (e.g., telephone, e-mail, letter) to understand the primary reason for this decision and record this information in the subject's chart and on the appropriate CRF pages. They may be considered withdrawn if they state an intention to withdraw, fail to return for visits, or become lost to follow-up for any other reason.

The investigator should discontinue study treatment for a given subject if, he/she believes that continuation would be detrimental to the subject's well-being.

The following are potential reasons for study treatment discontinuation:
1. Documented Disease Progression prior to Week 12.
2. Use of another investigational or marketed anti-cancer therapy prior to documented disease progression.
3. Development of a serious or intolerable AE that necessitates discontinuation at the discretion of the Investigator.
4. Loss to follow-up. The site must attempt to contact the subject and reschedule the missed visit as soon as possible and counsel the subject on the importance of maintaining the assigned visit schedule and ascertain whether the subject wishes to and/or should continue in the study.
5. Investigator's decision. At the discretion of the Investigator, when he/she believes continued participation is would be detrimental to the subject's well-being.
6. Protocol deviation that, in the opinion of the Sponsor and Investigator, warrants discontinuation from the study.
7. Subject is not compliant with the terms of the protocol.
8. Study Termination by the Sponsor

Subjects who completely discontinue both study drugs during the treatment period should be scheduled for EOT or Treatment Exit Visit within 14 days following the date study drug is permanently discontinued, at which time all of the assessments listed in the Time and Events Schedule (Appendix A) will be performed.

Subjects who discontinue the post-treatment follow-up period for any reason will not complete any follow-up assessments and will be followed for survival only.

### Niraparib

Subjects may continue protocol therapy on niraparib for up to 24 weeks or until one of the following criteria applies:
Disease progression
Serious or life-threatening adverse event
Severe noncompliance with protocol as judged by the Investigator and/or Sponsor
Subject decision to withdraw
Subject becomes pregnant
Subject is diagnosed with MDS or AML (as confirmed by a hematologist)
Investigator, Sponsor, and/or TESARO becomes aware of conditions or events that suggest a possible risk or hazard to subjects if the clinical study continues
   Subject Withdrawal from the Study

Once a subject is permanently discontinued from study drug, he or she will enter in to the 30 Day Follow Up, and later into Survival Follow Up, unless consent is withdrawn from the entire study or subject is lost to follow-up.

Please note, the subject must be withdrawn from the study drug if any of the following occur:
Pregnancy
Death
Withdrawal of consent

If the subject withdraws consent for disclosure of future information, the sponsor may retain and continue to use any data collected before such a withdrawal of consent.

If a subject withdraws from the study, he/she may request destruction of any samples taken and not tested, and the investigator must document this in the site study records.

Subjects discontinued from the study after the first dose of study drug will be considered, at a minimum, for safety analysis. Discontinued subjects will not be replaced.

A subject will be considered lost to follow-up if he or she repeatedly fails to return for scheduled visits and is unable to be contacted by the study site.

The following actions must be taken if a subject fails to return to the clinic for a required study visit:
The site must attempt to contact the subject and reschedule the missed visit as soon as possible and counsel the subject on the importance of maintaining the assigned visit schedule and ascertain whether the subject wishes to and/or should continue in the study.
Before a subject is deemed lost to follow up, the investigator or designee must make every effort to regain contact with the subject (where possible, 3 telephone calls and, if necessary, a certified letter to the subject's last known mailing address or local equivalent methods). These contact attempts should be documented in the subject's medical record.

Should the subject continue to be unreachable, he/she will be considered to have withdrawn from the study.

### Safety Management, Monitoring and Reporting

### AE Definition

An Adverse Event is any untoward medical occurrence in a subject or clinical study subject, temporally associated with the use of study intervention, whether or not considered related to the study intervention.

NOTE: An AE can therefore be any unfavorable and unintended sign (including an abnormal laboratory finding), symptom, or disease (new or exacerbated) temporally associated with the use of study intervention.

### Adverse Events Meeting the AE Definition

Any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the investigator (i.e., not related to progression of underlying disease).
Exacerbation of a chronic or intermittent pre-existing condition including either an increase in frequency and/or intensity of the condition.
New conditions detected or diagnosed after study drug administration even though it may have been present before the start of the study.
Signs, symptoms, or the clinical sequelae of a suspected drug-drug interaction.
Signs, symptoms, or the clinical sequelae of a suspected overdose of either study drug or a concomitant medication. Overdose per se will not be reported as an AE/SAE unless it is an intentional overdose taken with possible suicidal/self-harming intent. Such overdoses should be reported regardless of sequelae.
“Lack of efficacy” or “failure of expected pharmacological action” per se will not be reported as an AE or SAE. Such instances will be captured in the efficacy assessments. However, the signs, symptoms, and/or clinical sequelae resulting from lack of efficacy will be reported as AE or SAE if they fulfil the definition of an AE or SAE.

### Adverse Events NOT Meeting the AE Definition

Any Grade 3 or 4 clinically significant abnormal laboratory findings as per CTCAE v5.0 or other abnormal safety assessments which are associated with the underlying disease, unless judged by the investigator to be more severe than expected for the subject's condition.
The disease/disorder being studied or expected progression, signs, or symptoms of the disease/disorder being studied, unless more severe than expected for the subject's condition.
Medical or surgical procedure (e.g., debulking, endoscopy, appendectomy): the condition that leads to the procedure is the AE.
Situations in which an untoward medical occurrence did not occur (social and/or convenience admission to a hospital).
Anticipated day-to-day fluctuations of pre-existing disease(s) or condition(s) present or detected at the start of the study that do not worsen.

### Serious Adverse Events

### Serious Adverse Event Definition

If an event is not an AE per definition above, then it cannot be an SAE even if serious conditions are met (e.g., hospitalization for signs/symptoms of the disease under study, death due to progression of disease).

An SAE is defined as any untoward medical occurrence that, at any dose:
A. Results in death
B. Is life-threatening

The term ‘life-threatening’ in the definition of ‘serious’ refers to an event in which the subject was at risk of death at the time of the event. It does not refer to an event, which hypothetically might have caused death, if it were more severe.

C. Requires inpatient hospitalization or prolongation of existing hospitalization

In general, hospitalization signifies that the subject has been detained (usually involving at least an overnight stay) at the hospital or emergency ward for observation and/or treatment that would not have been appropriate in the physician's office or outpatient setting. Complications that occur during hospitalization are AEs. If a complication prolongs hospitalization or fulfills any other serious criteria, the event is serious. When in doubt as to whether “hospitalization” occurred or was necessary, the AE should be considered serious.

Hospitalization for elective treatment of a pre-existing condition that did not worsen from baseline is not considered an AE.
D. Results in persistent disability/incapacity

The term disability means a substantial disruption of a person's ability to conduct normal life functions.

This definition is not intended to include experiences of relatively minor medical significance such as uncomplicated headache, nausea, vomiting, diarrhea, influenza, and accidental trauma (e.g., sprained ankle) which may interfere with or prevent everyday life functions but do not constitute a substantial disruption.
E. Congenital anomaly/birth defect
F. Other situations (important medical event):

Medical or scientific judgment should be exercised in deciding whether SAE reporting is appropriate in other situations such as important medical events that may not be immediately life-threatening or result in death or hospitalization but may jeopardize the subject or may require medical or surgical intervention to prevent one of the other outcomes listed in the above definition. These events should usually be considered serious.

Examples of such events include invasive or malignant cancers, intensive treatment in an emergency room or at home for allergic bronchospasm, blood dyscrasias or convulsions that do not result in hospitalization, or development of drug dependency or drug abuse.

SAEs will be collected during the screening period of the study, once the ICF form has been completed, regardless of whether study intervention has been administered. SAEs will be recorded on forms provided by the sponsor (or its designee). SAEs occurring more than 30 days after the last dose of study treatment should be reported to the sponsor (or its designee) only if an investigator considers the SAE to be related to study treatment.

### Subject Death

Any death that occurs after the subject signs an informed consent to participate in the study up to 30 days after the last dose study treatment should be reported to the Sponsor.

Deaths due to any reason that occur within 30 days after the last dose of study treatment may be recorded as an SAE but will not be reported expeditiously to regulatory agencies unless required. In support of the study objective, overall survival, subject deaths that occur during the survival follow-up period will also be captured.

### Adverse Event of Special Interest

Adverse events of special interest (AESI) are defined as events (serious or non-serious) which are ones of scientific and medical concern specific to the sponsor's product or program, for which ongoing monitoring and rapid communication by the investigator to the sponsor may be appropriate. These AE terms may include but are not limited to the following: hypotension requiring treatment, dyspnea requiring bronchodilators, angioedema, generalized urticaria, infusion reactions, injection site reactions, and others to be identified through an ongoing review of the data. Such events may require further investigation in order to characterize and understand them.

Recording and Follow-Up of AE and/or SAE

### AE and SAE Recording

When an AE/SAE occurs, it is the responsibility of the investigator to review all documentation (e.g., hospital progress notes, laboratory reports, and diagnostics reports) related to the event.
The investigator will then record all relevant AE/SAE information in the CRF.
It is not acceptable for the investigator to send photocopies of the subject's medical records to OncoQuest Pharmaceuticals Inc. (or designee) in lieu of completion of the AE CRF page.
There may be instances when copies of medical records for certain cases are requested by OncoQuest Pharmaceuticals Inc (or designee). In this case, all subject identifiers, with the exception of the subject number, will be redacted on the copies of the medical records before submission to OncoQuest Pharmaceuticals Inc. (or designee).
The investigator will attempt to establish a diagnosis of the event based on signs, symptoms, and/or other clinical information. Whenever possible, the diagnosis (not the individual signs/symptoms) will be documented as the AE/SAE.

### Assessment of Severity

The investigator will make an assessment of severity for each AE and SAE reported during the study according to CTCAE v5.0. It is important to distinguish between serious and severe AEs. Severity is a measure of intensity whereas seriousness is defined by the criteria detailed herein. An AE of severe intensity may not necessarily be considered serious.

### Assessment of Causality

The investigator is obligated to assess the relationship between study intervention and each occurrence of each AE/SAE. A “reasonable possibility” of a relationship conveys that there are facts, evidence, and/or arguments to suggest a causal relationship, rather than a relationship cannot be ruled out. The investigator will use clinical judgment to determine the relationship. Alternative causes, such as underlying disease(s), concomitant therapy, and other risk factors, as well as the temporal relationship of the event to study intervention administration will be considered and investigated. The investigator will also consult the IB and/or Product Information, for marketed products, in his/her assessment.

For each AE/SAE, the investigator must document in the medical notes that he/she has reviewed the AE/SAE and has provided an assessment of causality. There may be situations in which an SAE has occurred, and the investigator has minimal information to include in the initial report to OncoQuest Pharmaceuticals Inc. (or designee). However, it is necessary that the investigator provides an assessment of causality for every event at the time of the initial transmission of the SAE data to the sponsor (or its designee). The investigator may change his/her opinion of causality in light of follow-up information and send a SAE follow-up report with the updated causality assessment. The causality assessment is one of the criteria used when determining regulatory reporting requirements.

The following definitions (Table 9) should be considered when evaluating the relationship of AEs and SAEs to the study intervention.

TABLE 10
Categories of Relationship to Study Intervention

| | |
|---|---|
| Related | The AE is known to occur with the study intervention, there is |
| | a reasonable possibility that the study intervention caused the |
| | AE, or there is a temporal relationship between the study |
| | intervention and event. Reasonable possibility means that |
| | there is evidence to suggest a causal relationship between the |
| | study intervention and the AE. |
| Not | There is not a reasonable possibility that the administration of |
| Related | the study intervention caused the event, there is no temporal |
| | relationship between the study intervention and event onset, |
| | or an alternate etiology has been established. |

### Assessment of Expectedness

The sponsor Medical Monitor (or designee) will be responsible for determining whether an AE or SAE is expected or unexpected. An AE or SAE will be considered unexpected if the nature, severity, or frequency of the event is not consistent with the risk information previously described in the IB for the study intervention. The sponsor Medical Monitor (or designee) will be the final arbiter for determining whether an AE or SAE is expected or unexpected.

### Follow-Up of AEs and SAEs

The investigator is obligated to perform or arrange for the conduct of supplemental measurements and/or evaluations as medically indicated or as requested by OncoQuest Pharmaceuticals Inc. (or designee) to elucidate the nature and/or causality of the AE or SAE as fully as possible. This may include additional laboratory tests or investigations, histopathological examinations, or consultation with other health care professionals.

If a subject dies during participation in the study or during a recognized follow-up period, the investigator will provide OncoQuest Pharmaceuticals Inc. (or designee) with a copy of any post-mortem findings including histopathology.

New or updated information will be recorded in the originally completed CRF.

The investigator will submit any updated SAE data to OncoQuest Pharmaceuticals Inc. (or designee) within 24 hours of receipt of the information.

### Reporting of SAEs

All SAE must be reported to the sponsor designee within 24 hours of first awareness of the investigator and designated pharmacovigilance team as indicated on SAE reporting form.

An SAE Report Form must be completed for any SAE including death due to any cause or death due to an unknown cause, whether or not considered study related, including those listed in the protocol or IB, and including an assessment of whether there is a reasonable possibility that the study intervention caused the event. The investigator will report all SAEs, which occur after ICF has been signed, during the study treatment period, or within 30 days following the final dose of the study intervention. Any SAE experienced after 30 days following the final dose should only be reported to the sponsor (or its designee) if the investigator suspects a causal relationship to the study treatment.

Follow-up to SAE will be reported in the same manner as the initial SAE report, within 24 hours of the investigator receiving the follow-up information. Recurrent episodes, complications, or progression of the initial SAE must be reported as follow-up to the original episode regardless of when it occurs. The follow-up information should describe whether the event has resolved or continues, if and how it was treated, and whether the subject continued or withdrew from study participation. An SAE occurring at a different time interval or otherwise considered completely unrelated to a previously reported one should be reported separately as a new event.

All SAEs will be followed until satisfactory resolution, or until the site investigator deems the event to be chronic or the subject is stable; including those who may be withdrawn due to serious or unexpected adverse experiences. Other supporting documentation of the event may be requested by the sponsor (or its designee) and should be provided as soon as possible.

If the SAE is not previously documented in the IB for either study drug and is thought to be related to the study treatment, the sponsor Medical Monitor (or designee) may urgently require further information from the investigator for Health Authority reporting. The sponsor (or its designee) may need to issue an “Investigator Notification Letter”, to inform all investigators involved in any study with the same drug that this SAE has been reported. Suspected Unexpected Serious Adverse Reactions (SUSARs) will be collected and reported to the FDA.

### Reporting of Pregnancy

All pregnancies must be reported to the sponsor designee within 24 hours of first awareness of the investigator and/or his staff via Pregnancy Report Form. Any subject who has a positive pregnancy test after signing ICF should have study intervention discontinued and be withdrawn from the study immediately. The investigator will report all pregnancies which occur after ICF has been signed, during the study treatment period, or within 30 days following the final dose of the study intervention. Pregnancy in and of itself is not regarded as an AE. A pregnancy will be considered as an SAE if the mother and/or the product of the pregnancy die, the pregnancy itself causes hospitalization or prolongation of an existing one, or the offspring results with a congenital anomaly or birth defect.

The investigator must follow-up on the status of the pregnancy to determine pregnancy outcome (including premature termination) and the status of mother and child. Follow-up pregnancy information must be reported to the sponsor designee within 24 hours of awareness. Pregnancy complications, spontaneous abortions, and/or elective terminations for medical reasons must also be reported. Possible relatedness to study drug must be assessed at the time of reporting.

When further information becomes available, the Pregnancy Report Form should be updated with all new information and reported immediately via the same contact information above. Additional information will be requested by the sponsor designee as necessary.

### Data Safety Monitoring Board

No formal Data Safety Monitoring Board (DSMB) will be instituted for this study.

### Safety Data Review Committee

A Safety Data Review Committee (SDRC), consisting of Investigators and Sponsor representatives, will be established to monitor safety during the study. On the basis of the available safety information from subjects, the SDRC will make recommendations to the Sponsor regarding continuation, modification or termination of the study. Of note, the final decision to suspend, amend or terminate the study remains with the Sponsor. The SDRC will be governed by a separate charter.

### Statistical Methods

Details of the statistical methods for this study will be documented in a Statistical Analysis Plan (SAP).

### Determination of Sample Size

A sample size of 10 subjects is estimated to provide adequate initial descriptive assessment of safety interactions for the combination therapy and also provide preliminary evidence of clinical and immune response patterns. The study should also provide signals to permit design estimates for future studies. The Sponsor may, after a review of the available safety and efficacy data from subjects, enroll up to 30 additional subjects for further evaluation in this study.

The decision to enroll additional subjects will be based on the following:
Increased early humoral response to infused antibody: ≥60% at week 7 (˜DAY42),
Disease Control Rate (DCR); 40% CR/PR/SD at Week 12.

### Populations for Analysis

The Intent-to-treat (ITT) population will include all enrolled subjects (defined as subjects who have received at least one dose of both study drugs). The ITT analysis set will serve as the primary analysis population for all efficacy endpoints and demographics.

The Per-protocol (PP) population will include all subjects who have baseline and post baseline efficacy measurements, and at least three infusions of oregovomab with no major protocol violations that could influence the assessment of efficacy. The PP population will be used as supportive for efficacy endpoints.

The Safety population will include all subjects who received any amount of study drug (niraparib, or oregovomab). The Safety population will be the primary analysis set for safety endpoints.

### Procedures for Handling Missing, Unused, and Spurious Data

No imputation of values for missing data will be performed, other than as described in this section. Further details on handling of missing data will be described in the statistical analysis plan.

For DCR, subjects who do not have 12-week efficacy assessment will be considered as censored at time 0. Additional censoring details will be described in the Statistical Analysis Plan.

For AEs, missing and partial dates will be handled as follows in determination of treatment-emergence. If partial dates are available, they will be used to assess if the AE occurred during the treatment period. AEs with completely missing start and end dates will be assumed to be treatment-emergent.

Missing AE severities will not be imputed and will be considered missing in any tabulations of AE severity. If an AE is missing a response to the question regarding relationship to treatment, the event will be considered related.

### General Methods

Baseline demographics, disease characteristics, safety parameters, laboratory and clinical data will be tabulated using descriptive statistics (mean, standard deviation, median, minimum, and maximum) for continuous variables and frequency counts and percentages for discrete variables. Kaplan-Meier estimation methods will be used for estimation of summary statistics for both PFS, DOR and OS. The 25th, 50th (median), and 75th percentiles with associated 2-sided 95% confidence intervals will be presented, as well as the proportion of censored subjects. All data will be provided in listings. All statistical analyses will be performed using SAS version 9.4 or later. No formal statistical testing will be performed.

### Analysis Time Points

An interim analysis will be performed when at least 10 evaluable subjects have completed the Week 12 visit. To be included in the interim analysis group of subjects, the following criteria must be met:
Subjects must have had at least 3 doses of oregovomab and at least 80% compliance with niraparib dose administration.
Subjects must have available data (screening and at least one post-treatment sample collected) for early humoral immune response at week 7 sample.
Subjects must have completed at least two imaging assessments.
Up to 5 subjects will be replaced if they do not fulfill the above criteria. Subjects who receive at least one dose of either study drug will not be replaced. A subject who is screened but does not receive either study drug may be replaced.

The DCR and ORR analysis will be done after last subject has had Week 24 visit plus 30-Day safety follow-up visit (LPLV). After last subject exits treatment period, final analysis will be completed, and final clinical study report (CSR) will be completed. The Sponsor reserves the option to complete a CSR Addendum to account for any long-term survival data, if available

### Statistical Analysis

### Disposition, Demographic, and Baseline Characteristics

A tabulation of subject disposition will be presented, including the number in each analysis population the number lost to follow-up, the number that withdrew prior to completing the study, and reason(s) for withdrawal.

Demographic and baseline characteristics will be summarized in a descriptive fashion. Data to be evaluated will include age, race, disease history and prior treatment. No formal statistical comparisons will be performed.

### Efficacy Analysis

The preliminary assessments of anti-tumor activity in this therapy combination include Disease Control Rate (CR/PR, and SD) and PFS. The disease control rate (CR, PR, or SD) and the best overall response (BOR) will be determined at Weeks 12 and 24 The BOR is defined as the best recorded assessment of CR and PR from the start of treatment until progression/recurrence as defined by RECIST 1.1. Data listings will show the response at each time point as well as the therapy administered after Week 12. An additional exploratory assessment of DCR for subjects receiving second line chemotherapy post Week 12, will be evaluated using Week 12 as the baseline for that assessment and listed separately.

Progression-free survival will be calculated from the date of first study treatment to the earliest date of disease progression as determined by the local radiologists/investigators or date of death. Subjects who have no documented progression and are still alive prior to the analysis will be censored on the date of last complete disease assessment according to RECIST v1.1 (prior to 2 or more missed assessments, if applicable). PFS will be listed for all subjects. Tumor assessments must be performed as per schedule regardless of delays or interruptions of study treatment.

Overall survival will be calculated from the date of first study drug treatment to the date of death. Subjects who are still alive at the time of analysis, or who dropout prior to study end, will be censored at the day they were last known to be alive.

### Analysis of Exploratory Markers of Activity

Exploratory markers will be described in the SAP. One prognostic factor, the baseline NMLR, will be associated with clinical parameters.

### Early Humoral Immune Response Analysis

Early humoral response to infused antibody will be assessed using a human anti-mouse antibody kit. Values at baseline and all subsequent measured timepoints will be summarized and tabulated. In addition, the incidence of patients who have increased HAMA greater than 74 ng/ml, relative to baseline, will be calculated. Also, the ratio of Week 7 to baseline values, early humoral response ratio (EHRR), will be calculated for each patient and associated with clinical parameters.

### Cellular Immune Response Analysis

Samples for future evaluation of tumor specific cellular immunity and immune phenotyping will be tissue banked; however, no analysis of this data is anticipated in the primary analysis and summary report of this study. A future supplemental report and analysis will be prepared when these exploratory studies are completed.

### Safety Analysis

Safety will be assessed by Treatment Emergent Adverse Events (TEAEs) as per CTCAE v5.0, physical examinations, laboratory assessments, and vital signs.

### Adverse Events

Adverse events will be coded using MedDRA version 22 or higher for purposes of summarization and displayed in tables and listings using System Organ Class (SOC) and Preferred Term.

Analyses of AEs will be performed for those events that are considered treatment-emergent, where treatment-emergent is defined as any AE that occurs after the first dose of treatment through 30 days following last dose or any event considered drug-related by the Investigator, or any event that is present at baseline but worsens in intensity.

Adverse events are summarized by subject incidence rates, therefore, in any tabulation, a subject contributes only once to the count for a given AE (preferred term). Separate tabulations will be produced for serious AEs (SAEs), AEs that lead to discontinuation, AEs assessed by the Investigator as related to treatment (possibly, probably, or definitely drug-related), and AEs of at least CTCAE Grade 3 severity. Infusion related reactions (IRRs), hypersensitivity reactions (HSRs) and Adverse Events of Special Interest (AESIs) will also be tabulated. The AE terms used for AESI will be listed in the SAP.

No formal hypothesis-testing of AE incidence rates will be performed.

Adverse events of special interest (AESI) will require ongoing monitoring and rapid communication by the investigator to the sponsor. The AESI terms may include but are not limited to the following: hypotension requiring treatment, dyspnea requiring bronchodilators, angioedema, generalized urticaria, infusion reactions, injection site reactions, and others to be identified through an ongoing review of the data.

All AEs (treatment-emergent and post-treatment) will be listed in subject data listings. Separate listings will be provided for the following: subject deaths, AESIs, SAEs, and AEs leading to discontinuation.

### Study Drugs Exposure

Study drug exposure will be summarized for oregovomab and niraparib, including number of doses of each drug, number of dose reductions or dose holds/interruptions, and overall study drug exposure, calculated as (last dose date of any study drug—first dose date of any study drug +1)/30.4375.

### Laboratory Data, Vital Signs, and Physical Examinations

Clinical laboratory values will be expressed using conventional SI units. Severity of select clinical lab measures will be determined using CTCAE criteria (e.g., those measures that have a corresponding CTCAE grade classification). Shift tables that present changes from Baseline to worst on-study relative to CTCAE classification ranges will be produced.

The actual value and change from Baseline to each on-study evaluation will be summarized for laboratory parameters and vital signs. Any additional safety measurements, including physical examination will be listed by subject.

### Concomitant Medications

Concomitant medications will be coded using the WHO Drug Dictionary. Results will be tabulated by Anatomic Therapeutic Class (ATC) and preferred term.

Concomitant medications will be tabulated, where any medications that were not discontinued prior to the first dose of treatment will be included. If an end date is missing or the medication is ongoing at the time of first dose, the medication will be considered concomitant.

### Administrative Requirements

### Good Clinical Practice Statement

By mutual consent, OncoQuest Pharmaceuticals Inc. and Investigator(s) agree to adhere to the protocol. Any change in the study must be agreed upon in writing before initiation of the change by the Investigator (s) and OncoQuest Pharmaceuticals Inc.

The Investigator(s) also agree(s) to conduct the study in accordance with regulations of the United States Food and Drug Administration (FDA) and other relevant health authorities and with the principles of Good Clinical Practice (GCP).

### Ethical Considerations

The study will not begin until all regulatory requirements have been fulfilled. The study will be conducted in accordance with the protocol, the most recent version of the Declaration of Helsinki, and the ICH tripartite guidelines for GCP as adopted by FDA in 1996.

### Protocol Review

This protocol must be reviewed and approved by the appropriate Institutional Review Board (IRB)/Independent Ethics Committee (IEC) where the study is to be conducted. The Investigators are responsible for obtaining and maintaining approvals (at intervals not less than once a year) of the study by the IRB/IEC. Written approval from the IRB/IEC must be provided to OncoQuest Pharmaceuticals Inc. before shipment of the study drug can be authorized.

The Investigator will submit all changes, amendments and revisions to OncoQuest Pharmaceuticals Inc., or its designee, and the IRB/IEC. Any change to the protocol that affects subject selection, safety, or changes in the conduct of the study will require written approval from OncoQuest Pharmaceuticals Inc. and the IRB/IEC before implementing the change. A protocol change intended to eliminate an apparent immediate hazard may be implemented immediately, but the IRB/IEC must still be notified.

### Informed Consent Review

Written consent is required in this study. If the subject is legally incompetent (i.e., a minor or mentally incapacitated), the written consent of a parent, legal guardian or legal representative must be obtained, and such consent must be witnessed.

The Investigator also thereby agrees that OncoQuest Pharmaceuticals Inc. and the IRB/IEC will approve all subject informed consent forms before the study is initiated. The Investigator or designee will obtain informed consent and document this process for all subjects enrolled in this study by noting the date and time of consent in the source document. Informed consent must be obtained prior to initiating any study-related screening procedures.

The Informed Consent Form will explain to potential subjects the nature of this study and its purposes, risks and duration. Subjects will be advised who to contact for advice regarding the study, and what to do in the event of an adverse reaction during the study. The informed consent form will state that representatives from OncoQuest Pharmaceuticals Inc. or government drug regulatory agencies may review the study data in their files.

Once a subject agrees to participate in the study, the subject will sign the approved consent form. The original signed consent form should be placed in the subject's permanent file. A copy should be kept on file by the Investigator and another given to the subject for reference. In the event that any study procedures or safety information changes and impacts the subject's participation, the subject must be re-consented to the most current version of the ICF(s) during their participation in the study.

### Financial Disclosure

Investigators and sub-investigators will provide the sponsor with sufficient, accurate financial information as requested to allow OncoQuest Pharmaceuticals Inc., or its designee, to submit complete and accurate financial certification or disclosure statements to the appropriate regulatory authorities. Investigators are responsible for providing information on financial interests during the course of the study and for 1 year after completion of the study.

### Sponsor Monitoring

Throughout the course of the study, individuals from OncoQuest Pharmaceuticals Inc. or its designee will visit the study site to monitor the progress of the study. These visits will be for the purpose of monitoring adherence to the protocol, completeness of data collection and recording, and investigational drug accountability.

By signing this protocol, the Investigator agrees that, within local regulatory restrictions and institutional and ethical considerations, OncoQuest Pharmaceuticals Inc. or its designee may consult and/or copy data from source documents (e.g., nuclear medicine reports, laboratory reports, ECG, x-rays, diagnostic imaging reports, workbooks and subject's medical records) in order to verify CRF data.

It is expected that the Investigator and/or other appropriate staff are available on the day of the visit in case any questions arise.

### Study Drug Accountability

Both drugs will be kept in a secure location and will be accessible only to the pharmacist or research staff member, who is qualified to prepare and dispense medication, and who is authorized to dispense the study drugs.

The pharmacist or qualified research staff member will maintain inventory records for both study drugs. The records will include details of materials received, the date dispensed, the subject identification number and initials of subject receiving the dose.

At the conclusion or termination of the study, OncoQuest Pharmaceuticals Inc. (or its designee) and the study pharmacist or qualified research staff member will conduct a final study drug supply inventory. All unused study drug may be destroyed at the site according to institutional guidelines and a Certificate of study drug Supply Destruction Form (or similar form) must be completed.

Destruction of any remaining study drug may not occur until final accountability monitoring.

### Case Report Forms and Source Documentation

Subject data will be recorded on an Electronic Data Capture (EDC) case report form (CRF). The CRF are to be reviewed by the Investigator for completeness and accuracy. The Investigator must sign each subject's CRF using a validated electronic signature.

The Investigator will allow designated OncoQuest Pharmaceuticals Inc. representatives and regulatory bodies to have direct access to the source documents to verify the data reported in the CRFs. Source documents are the originals of any documents used by the Investigator or hospital/institution that allow verification of the existence of the subject and substantiate the integrity of the data collected during the study.

Source documents should be available to support all the data recorded in the CRF, unless this is otherwise specified in the CRF. However, data reported on the CRF or entered in the CRF that are transcribed from source documents must be consistent with the source documents or the discrepancies must be explained.

### Protocol Deviations

Any deviation from the protocol will be recorded as a protocol deviation.

OncoQuest Pharmaceuticals Inc. and delegated external vendors (as applicable) will follow ICH E3 Structure and Content of Clinical Study Report instructions as it relates to the identification, tracking, categorization and reporting of Protocol Deviations.

Important protocol deviations are a subset of protocol deviations that might significantly affect the completeness, accuracy, and/or reliability of the study data or that might significantly affect a subject's rights, safety, or well-being. Important protocol deviations may include, but are not limited to, the following:
subjects who entered the study even though they did not satisfy the entry criteria
subjects who developed withdrawal criteria during the study but were not withdrawn
subjects who received the wrong treatment or incorrect dose
subjects who received an excluded concomitant treatment

Individual subjects with these protocol deviations will be listed in the final Clinical Study Report by country and site.

### Records Retention

Essential documents (written and electronic) should be retained for a period of not less than fifteen (15) years from the completion of the Clinical Trial unless OncoQuest Pharmaceuticals Inc., or its designee, provides written permission to dispose of them or, requires their retention for an additional period of time because of applicable laws, regulations and/or guidelines.

The investigator/institution should maintain the trial documents as specified in Essential Documents for the Conduct of a Clinical Trial and as required by applicable regulations and/or guidelines. The investigator/institution should take measures to prevent accidental or premature destruction of these documents.

### Confidentiality of Study Documents and Patient Records

Each participating site will maintain appropriate medical and research records for this study, in compliance with Section 4.9 of the ICH E6 GCP, and regulatory and institutional requirements for the protection of confidentiality of subjects.

By signing this protocol, the Investigator affirms to OncoQuest Pharmaceuticals Inc. that the information furnished by OncoQuest Pharmaceuticals Inc. will be maintained in confidence and will divulge such information to an IRB/IEC under an appropriate understanding of confidentiality with such a committee.

### Data Quality Assurance

All subject data relating to the study will be recorded in the CRF unless transmitted to the sponsor or designee electronically (e.g., laboratory data). The investigator is responsible for verifying that data entries are accurate and correct by physically or electronically signing the CRF.
The investigator must maintain accurate documentation (source data) that supports the information entered in the CRF.
The investigator must permit study-related monitoring, audits, IRB/IEC review, and regulatory agency inspections and provide direct access to source data documents.
Monitoring details describing strategy (e.g., risk-based initiatives in operations and quality such as Risk Management and Mitigation Strategies and Analytical Risk-Based Monitoring), methods, responsibilities and requirements, including handling of noncompliance issues and monitoring techniques (central, remote, or on-site monitoring) are provided in the Monitoring Plan.
OncoQuest Pharmaceuticals Inc. or designee is responsible for the data management of this study including quality checking of the data.
OncoQuest Pharmaceuticals Inc. assumes accountability for actions delegated to other individuals (e.g., Contract Research Organizations).
Study monitors will perform ongoing source data verification to confirm that data entered into the CRF by authorized site personnel are accurate, complete, and verifiable from source documents; that the safety and rights of subjects are being protected; and that the study is being conducted in accordance with the currently approved protocol and any other study agreements, ICH GCP, and all applicable regulatory requirements.

### Data Protection

All information about the subject's data disclosed to the Investigator by the sponsor or persons assigned by the Sponsor will be treated as strictly confidential. Subjects will be assigned a unique identifier by OncoQuest Pharmaceuticals Inc. (or Designee). Any subject records or datasets that are transferred to the OncoQuest Pharmaceuticals Inc. by third-party vendors will contain the identifier only; subject names or any other information which would make the subject identifiable will not be transferred.

The subject will be informed that any personal study-related data will be used by the sponsor in accordance with local data protection laws. The level of disclosure will be explained to the subject.

The subject will be informed that her medical records may be examined by Clinical Quality Assurance auditors or other authorized personnel appointed by the sponsor, by appropriate IRB/IEC members, and by inspectors from regulatory authorities.

### Site Termination Criteria

The sponsor designee reserves the right to close the study site or terminate the study at any time for any reason at the sole discretion of the sponsor.

A study site is considered closed when all required documents and study supplies have been collected and a study-site closure visit has been performed.

The investigator may initiate study-site closure at any time, provided there is reasonable cause and sufficient notice is given in advance of the intended termination.

Reasons for the early closure of a study site by the sponsor or investigator may include but are not limited to:
Failure of the investigator to comply with the protocol, the requirements of the IRB/IEC or local health authorities, the sponsor's procedures, or GCP guidelines
Inadequate recruitment of subjects by the investigator
Discontinuation of further study drug development

If, after 6 months from the initiation of the protocol, unforeseen circumstances have prevented the accrual of subjects into the study at a specific site, the Investigator or OncoQuest Pharmaceuticals Inc. may request that the study site be closed.

### Publication Policy

The results of this study may not be published or presented at scientific meetings without expressed permission of OncoQuest Pharmaceuticals Inc. If this is foreseen, the investigator agrees to submit all manuscripts or abstracts to the sponsor before submission. This allows the sponsor to protect proprietary information and to provide comments.

OncoQuest Pharmaceuticals Inc. will comply with the regulatory requirements for publication of study results. Authorship will be determined by OncoQuest Pharmaceuticals Inc. and in line with International Committee of Medical Journal Editors authorship requirements.

It is noted that publication of a summary of the results of the study in the customary manner is permissible and will be consistent with the data protection guidelines. Subject confidentiality will be maintained by the Investigator and OncoQuest Pharmaceuticals Inc. in accordance with HIPAA and other regulatory guidelines.

TABLE 1
Time and Events Schedule
APPENDIX A: TIME AND EVENTS SCHEDULE

| Procedure | | | | | | | | | | | |
|---|---|---|---|---|---|---|---|---|---|---|---|
| | | | | | | | | | EOT/ | 30 Day | |
| Protocol | | Week | Week | Week | Week | Week | Week | Week | Early | Safety | |
| Section | Screening | 1 | 4 | 7 | 12 | 16 | 20 | 24 | Term | F/U | LTFU* |

Days

| | | | | | | | |
|---|---|---|---|---|---|---|---|
| −28-0 | Day 1 | Day 22 | Day 43 | Day 78 | Day 112 | Day 134 | Day 162 |

Window (days), unless otherwise specified

| | | | | | | | | | | |
|---|---|---|---|---|---|---|---|---|---|---|
| −4 to −0 | | | | | | | | | | |
| weeks | N/A | ±3 | ±3 | ±3 | ±3 | ±3 | ±3 | ±14 | ±3 | ±7 |

SCREENING

| | | |
|---|---|---|
| Written | 7.1.1 | x |
| Informed Consent | | |
| Inclusion/Exclusion | 0/0 | x |
| Criteria | | |

SUBJECT HISTORY

| | | |
|---|---|---|
| Demography | 7.1.2 | x |
| Past/Current Medical | 7.1.3 | x |
| History | | |
| Relevant Cancer | 7.1.4 | x |
| Diagnosis/History | | |
| Prior Anti-Cancer | 7.1 | x |
| Therapies | | |
| FIGO Staging | 7.1.5 | x |
| BRCA1/BRCA2/HRD | 7.1.8 | x |
| Status (historical) | | |

PHYSICAL HEALTH EXAMINATIONS

| | | | | | | | | | | | |
|---|---|---|---|---|---|---|---|---|---|---|---|
| Physical Exam | 7.2.2 | x | x | x | x | x | x | x | x | x | x |
| ECOG | 7.2.3 | x | x | x | x | x | x | x | x | x | x |
| Height/Weight, | 7.2.1 | x | x | x | x | x | x | x | x | x | |
| including BMI | | | | | | | | | | | |
| Vital Signs (BP, | 7.2.1 | x | x | x | x | x | x | x | x | x | x |
| Temp, Pulse) | | | | | | | | | | | |

LABORATORY ASSESSMENTS

| | | | | | | | | | | | |
|---|---|---|---|---|---|---|---|---|---|---|---|
| Hematology, | 7.2.4 | x | x | x | x | x | x | x | x | x | x |
| Biochemistry | | | | | | | | | | | |
| CA-125 | 7.3.1.1 | x | | x | | x | | | x | | |
| HAMA | 7.3.1 | x | | | x | | | x | x | | |
| TSH (T3, T4 if | 7.2.4 | x | | | | | | | | | |
| TSH abnormal) | | | | | | | | | | | |
| Urinalysis | 7.2.4 | x | x | x | x | x | x | x | x | | |
| Serum Pregnancy | 7.2.4 | x | | | | | | | | | |
| Test B-HCG | | | | | | | | | | | |
| (if childbearing) | | | | | | | | | | | |
| Urine Pregnancy Test | 7.2.4 | | x | x | x | x | | x | | x | |
| ECG | 7.2.6 | x | | | | x | | | x | x | |
| Immune Response | 7.3.1 | x | | | | x | | | x | | |
| Parameters | | | | | | | | | | | |

STUDY TREATMENT

| | | | | | | |
|---|---|---|---|---|---|---|
| Oregovomab IV | 6.3 | x | x | x | x | x |
| Administration | | | | | | |

| | | | |
|---|---|---|---|
| Niraparib PO | 6.3 | 200 mg or | If appropriate, or |
| Administration | | 300 mg daily | treatment of choice |

RADIOLOGICAL EVALUATIONS

| | | | | |
|---|---|---|---|---|
| Imaging Evaluation | 7.2.6 | x | x | x |
| (CT/MRI) as per RECIST | | | | |
| v1.1 | | | | |

SAFETY ASSESSMENTS

| | | | | | | | | | | | |
|---|---|---|---|---|---|---|---|---|---|---|---|
| Prior/Concomitant | 6.8 | x | x | x | x | x | x | x | x | x | |
| Medications | | | | | | | | | | | |
| Anti-Cancer therapies | 6.9 | | | | | x | x | x | x | x | x |
| after discontinuing | | | | | | | | | | | |
| niraparib | | | | | | | | | | | |
| Adverse Events | 9.1 | x | x | x | x | x | x | x | x | x | |
| Phone Call* | | | | | | | | | | x | x |

*If a subject cannot come in for their 30-Day Safety F/U or any of the Long term Follow Up visits, a phone call may replace the clinic visit. In such cases, physical health examinations and required lab assessments will NOT need to be performed.

APPENDIX B: International Federation of Gynecologists and
Obstetricians (FIGO) Staging for Cancer of the ovary, fallopian tube,
and peritoneum

| FIGO | |
|---|---|
| Stage | Description |
| I | Tumor limited to the ovaries or fallopian tube(s) |
| IA | Tumor limited to 1 ovary (capsule intact) or fallopian tube; no |
| | tumor on ovarian or fallopian tube surface; no malignant cells in |
| | the ascites or peritoneal washings |
| IB | Tumor limited to both ovaries (tablets intact) or fallopian tubes; |
| | no tumor on ovarian or fallopian tube surface; no malignant cells |
| | in the ascites or peritoneal washings |
| IC | Tumor limited to 1 or both ovaries or fallopian tubes, with any |
| | of the following: |
| | IC1: Surgical spill |
| | IC2: Capsule ruptured before surgery or tumor on ovarian or |
| | fallopian tube surface |
| | IC3: Malignant cells in the ascites or peritoneal washings |
| II | Tumor involves 1 or both ovaries or fallopian tubes with pelvic |
| | extension (below pelvic brim) or peritoneal cancer |
| IIA | Extension and/or implants on uterus and/or fallopian tubes |
| | and/or ovaries |
| IIB | Extension to other pelvic intraperitoneal tissues |
| III | Tumor involves 1 or both ovaries or fallopian tubes, or peritoneal |
| | cancer, with cytologically or histologically confirmed spread to |
| | the peritoneum outside the pelvis and/or metastasis to the |
| | retroperitoneal lymph nodes |
| IIIA | IIIA1: Positive retroperitoneal lymph nodes only (cytologically |
| | or histologically proven): |
| | IIIA1(i) Metastasis up to 10 mm in greatest dimension |
| | IIIA1(ii) Metastasis more than 10 mm in greatest dimension |
| | IIIA2: Microscopic extra-pelvic (above the pelvic brim) |
| | peritoneal involvement with or without positive retroperitoneal |
| | lymph nodes |
| IIIB | Macroscopic peritoneal metastasis beyond the pelvis up to 2 cm |
| | in greatest dimension, with or without metastasis to the |
| | retroperitoneal lymph nodes |
| IIIC | Macroscopic peritoneal metastasis beyond the pelvis more than |
| | 2 cm in greatest dimension, with or without metastasis to the |
| | retroperitoneal lymph nodes (includes extension of tumor to |
| | capsule of liver and spleen without parenchymal involvement |
| | of either organ) |
| IV | Distant metastasis excluding peritoneal metastases |
| | IVA: Pleural effusion with positive cytology |
| | IVB: Parenchymal metastases and metastases to extra-abdominal |
| | organs (including inguinal lymph nodes and lymph nodes outside |
| | of the abdominal cavity) |

Reference: Berek et al., 2018

### REFERENCES

1. Aghajanian C, et al. OCEANS: a randomized, double-blind, placebo-controlled phase III trial of chemotherapy with or without bevacizumab in patients with platinum-sensitive recurrent epithelial ovarian, primary peritoneal, or fallopian tube cancer. J Clin Oncol. 2012; 30(17):2039-45
2. American Cancer Society. Cancer Facts & FIGS. 2018. Atlanta: American Cancer Society; 2018.
3. Battaglia A, Fattorossi A, Madiyalakan M R, Mahnke Y D, Nicodemus C. et al. Translational immune correlates of indirect antibody immunization in a randomized phase II study using scheduled combination therapy with carboplatin/paclitaxel plus oregovomab in ovarian cancer patients. Cancer Immunol Immunother. 2020 March; 69(3):383-397.
4. Berek J, Taylor P, McGuire W, Smith L M, Schultes B, Nicodemus C F. Oregovomab Maintenance Monoimmunotherapy Does Not Improve Outcomes in Advanced Ovarian Cancer. J Clin Oncol 2009; 27:418-425.
5. Berek J S, Taylor P T, Gordon A, Cunningham M J, Finkler N, Orr, Jr. J, et al. Randomized, Placebo-Controlled Study of Oregovomab for Consolidation of Clinical Remission in Patients with Advanced Ovarian Cancer. J Clin Oncol 2004; 22:3507-3516.
6. Berek J S, Matulonis, U A, et al: Ann Oncol, Safety and dose modification for patients receiving niraparib. 2018 Aug. 1; 29(8):1784-1792
7. Braly P, Nicodemus C F, Chu C, et al. The Immune adjuvant properties of front-line carboplatin-paclitaxel: a randomized phase 2 study of alternative schedules of intravenous oregovomab chemoimmunotherapy in advanced ovarian cancer. J Immunother. 2009 January; 32(1):54-65
8. Brewer M, Braly P, Madiyalakan M, Bayever E, Nicodemus C, et al. Front-line chemo-immunotherapy with carboplatin-paclitaxel using oregovomab indirect immunization in advanced ovarian cancer: A randomized phase II study. Gynecol Oncol. 2020 March; 156(3):523-529.
9. Burger R A, Brady M F, Bookman M A, et al. Incorporation of bevacizumab in the primary treatment of ovarian cancer. N Engl J Med 2011; 365:2473-83.
10. Cancer Genome Atlas Research N. Integrated genomic analyses of ovarian carcinoma. Nature. 2011; 474(7353):609-615.
11. Coleman R L, et al. Bevacizumab and paclitaxel-carboplatin chemotherapy and secondary cytoreduction in recurrent, platinum-sensitive ovarian cancer (NRG Oncology/Gynecologic Oncology Group study GOG-0213): a multicentre, open-label, randomised, phase 3 trial. Lancet Oncol. 2017 June; 18(6):779-791
12. De Lorenzo S B, Patel A G, Hurley R M, Kaufmann S H. The Elephant and the Blind Men: Making Sense of PARP Inhibitors in Homologous Recombination Deficient Tumor Cells. Front Oncol. 2013; 3:228.
13. Dietrich B, Srinivas S. Urothelial carcinoma: the evolving landscape of immunotherapy for patients with advanced disease. Res Rep Urol. 2018; 10: 7-16.
14. E A Eisenhauer et al. New response evaluation criteria in solid tumours: Revised RECIST guideline (version 1.1). EJC 45 (2009) 228-247
15. Fellner C. Ipilimumab prolongs survival in advanced melanoma. Pharm & Ther 2012; 37:503-540.
16. Fendrick J L, Staley K A, Gee M A, McDougald S R, Quirk J G Jr, O'Brien T J. Characterization of CA125 synthesized by human epithelial amnion WISH cell line. Tumor Biol 1993; 14:310-318.
17. Fong P C, Boss D S, Yap T A, et al. Inhibition of poly(ADP-ribose) polymerase in tumors from BRCA mutation carriers. N Engl J Med. 2009; 361(2):123-13
18. Friedlander et al., A phase 1b study of the anti-PD-1 monoclonal antibody BGB-A317 (A317) in combination with the PARP inhibitor BGB-290 (290) in advanced solid tumors. J Clin Oncol 36, 2018 (suppl 5S; abstr 48)
19. Gelmon K A, Tischkowitz M, Mackay H, et al. Olaparib in patients with recurrent high-grade serous or poorly differentiated ovarian carcinoma or triple-negative breast cancer: a phase 2, multicentre, open-label, non-randomised study. Lancet Oncol. 2011; 12(9):852-861.
20. J. Hamanishi, M. Mandai, T. Ikeda, M. Minami, A. Kawaguchi, T. Murayama, M. Kanai, Y. Mori, S. Matsumoto, S. Chikuma, et al. Safety and antitumor activity of anti-PD-1 antibody, Nivolumab, in patients with platinum-resistant ovarian cancer J. Clin. Oncol., 33 (2015), pp. 4015-4022
21. Jing w, et al., STING agonist inflames the pancreatic cancer immune microenvironment and reduces tumor burden in mouse models, Journal for ImmunoTherapy of Cancer 2019: Article number:115
22. Jones P, Wilcoxen K, Rowley M, Toniatti C. Niraparib: A Poly(ADP-ribose) Polymerase (PARP) Inhibitor for the Treatment of Tumors with Defective Homologous Recombination. J Med Chem. 2015; 58(8):3302-3314.
23. K. Matsuo, Y. G. Lin, L. D. Roman, A. K. Sood. Overcoming platinum resistance in ovarian carcinoma Expert Opin. Investig. Drugs, 19 (2010), pp. 1339-1354
24. Konstantinopoulos P A, Waggoner S, Vidal G A, et al. Single-Arm Phases 1 and 2 Trial of Niraparib in Combination With Pembrolizumab in Patients With Recurrent Platinum-Resistant Ovarian Carcinoma. JAMA Oncol. 2019 June; 1048.
25. Kummar S, Ji J, Morgan R, et al. A Phase I study of veliparib in combination with metronomic cyclophosphamide in adults with refractory solid tumors and lymphomas. Clin Cancer Res. 2012; 18(6):1726-173
26. Mirza M R, et al, Niraparib Maintenance Therapy in Platinum-Sensitive, Recurrent Ovarian Cancer. N Engl J Med. 2016; 35(22):2154
27. Moore K N, et al., Niraparib monotherapy for late-line treatment of ovarian cancer (QUADRA): a multicentre, open-label, single-arm, phase 2 trial. Lancet Oncol. 2019 May; 20(5):636-648
28. Motzer R J, Escudier B, McDermott D F, George S, Hammers H H, Srinivas S, et al. Nivolumab versus everolimus in advanced renal-cell carcinoma. N Engl J Med 2015; 373:1803-1813.
29. Msaouel P, Masserelli E. Immune checkpoint therapy in head and neck cancers. Cancer J 2016; 22:108-116.
30. Oken M, Creech R, Tormey D, et al. Toxicity and response criteria of the Eastern Cooperative Oncology Group. Am J Clin Oncol. 1982; 5:649-655.
31. Overrman, M J, McDermott, R, Leach, J L, et al. Nivolumab in patients with metastatic DNA mismatch repair-deficient or microsatellite instability-high colorectal cancer (CheckMate 142): an open-label, multicentre, phase 2 study. Lancet Oncol 2017; 18:1182-1191.
32. Oza A M, Matulonis U A, Malander S, Hudgens S, Sehouli J, Del Campo J M, Mirza M R, et al., Quality of life in patients with recurrent ovarian cancer treated with niraparib versus placebo (ENGOT-OV16/NOVA): results from a double-blind, phase 3, randomised controlled trial. Lancet Oncol. 2018; 19(8):1117.
33. Pardoll D M. The blockade of immune checkpoints in cancer immunotherapy. Nat Rev Cancer 2012; 12:252-264.
34. Perren T J, Swart A M, Pfisterer J, et al. A phase 3 trial of bevacizumab in ovarian cancer. N Engl J Med 2011; 365:2484-96Jiang X, Pissaloux D, De La Fouchardiere C, et al. The sum of gains and losses of genes encoding the protein tyrosine kinase targets predicts response to multi-kinase inhibitor treatment: Characterization, validation, and prognostic value. Oncotarget 2015; 6:26388-99.
35. Pignata S, et al: Randomized Controlled Trial Testing the Efficacy of Platinum-Free Interval Prolongation in Advanced Ovarian Cancer: The MITO-8, MaNGO, BGOG-Ov1, AGO-Ovar2.16, ENGOT-Ov1, GCIG Study. J Clin Oncol. 2017; 35(29):3347
36. Pilié P G, Gay C M, Byers L A, O'Connor M J, Yap T A. PARP Inhibitors: Extending Benefit Beyond BRCA-Mutant Cancers. Clin Cancer Res. 2019 Jul. 1; 25(13):3759-3771.
37. Pujade-Lauraine E, Hilpert F, Weber B, et al. Bevacizumab combined with chemotherapy for platinum-resistant recurrent ovarian cancer: The AURELIA open-label randomized phase III trial. J Clin Oncol. 2014 Dec. 10; 32(35):4025
38. Sharma P, Allison J P. The future of immune checkpoint therapy. Science 2015; 348:55-61.
39. Timms K M, Abkevich V, Hughes E, et al. Association of BRCA1/2 defects with genomic scores predictive of DNA damage repair deficiency among breast cancer subtypes. Breast Cancer Res. 2014; 16(6):475.
40. Turner N, Tutt A, Ashworth A. Hallmarks of ‘BRCAness’ in sporadic cancers. Nat Rev Cancer. 2004; 4(10):814-819.
41. Turner N C, Ashworth A. Biomarkers of PARP inhibitor sensitivity. Breast Cancer Res Treat. 2011; 127(1):283-286.
42. Yin B W T, Dnistrian A, Lloyd K O. Ovarian cancer antigen CA125 is encoded by the MUC16 mucin gene. Int J Cancer 2002; 98:737-740.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While preferred embodiments have been described above and illustrated in the accompanying drawings, are evident to those skilled in the art that modifications may be made without departing from this disclosure. Such modifications are considered as possible variants comprised in the scope of the disclosure.

1. A method for improving likelihood of survival in a stage III-IV ovarian cancer patient, the method comprising at least steps (a) and (b):
(a) administering daily to a stage III-IV ovarian cancer patient a therapeutically effective dose of Niraparib, for at least 12 weeks;
(b) administering to the patient a therapeutically effective dose of monoclonal antibody mAb-B43.13 during week 1, week 4, week 7, week 12 and week 20 of a treatment period,
wherein step (a) is performed first and step (b) is performed second,
thereby increasing the patient's likelihood of survival in comparison with a control patient who has been diagnosed with stage III-IV ovarian cancer and has received standard of care chemotherapy treatment.

2. The method of claim 1, wherein the therapeutically effective dose of Niraparib is from about 200 to about 300 mg, or is 200 mg or is 300 mg.

3. The method of claim 2, wherein the therapeutically effective dose of Niraparib is orally administered.

4. The method of claim 1, wherein the therapeutically effective dose of monoclonal antibody mAb-B43.13 is administered on day 1 of the week 1, week 4, week 7, week 12 and week 20 of the treatment period.

5. The method of claim 1, wherein the treatment period is 24 weeks.

6. The method of claim 1, wherein 2 mg of mAb-B43.13 is administered

7. The method of claim 1, wherein mAb-B43.13 is administered in a volume of 50 ml by a 20-minute infusion.

8. A therapeutic agent for use in inhibiting stage III-IV ovarian cancer tumor growth in a patient, the therapeutic agent comprising mAb-B43.13 (oregovomab) and a parp inhibitor;
wherein said parp inhibitor is Niraparib;
wherein said therapeutic agent is used according to the following schedule:
(a) daily use by a stage III-IV ovarian cancer patient of a therapeutically effective dose of Niraparib, for at least 12 weeks;
(b) use by the patient of a therapeutically effective dose of monoclonal antibody mAb-B43.13 during week 1, week 4, week 7, week 12 and week 20 of a treatment period,
wherein step (a) is first and step (b) is second,
thereby increasing the patient's likelihood of survival in comparison with a control patient who has been diagnosed with stage III-IV ovarian cancer and has received standard of care chemotherapy treatment.

9. The therapeutic agent of claim 8, wherein the therapeutically effective dose of Niraparib from about 200 to about 300 mg, or is 200 mg or is 300 mg.

10. The therapeutic agent of claim 9, wherein the therapeutically effective dose of Niraparib is provided through oral use.

11. The therapeutic agent of claim 8, wherein the therapeutically effective dose of monoclonal antibody mAb-B43.13 is used on day 1 of the week 1, week 4, week 7, week 12 and week 20 of the treatment period.

12. The therapeutic agent of claim 8, wherein the treatment period is 24 weeks.

13. The therapeutic agent of claim 8, wherein 2 mg of mAb-B43.13 is used.

14. The therapeutic agent of claim 8, wherein mAb-B43.13 is used in a volume of 50 ml by a 20-minute infusion.

15.-21. (canceled)
